(19) United States
(12) Patent Application Publication
(10) Pub. No.: US 2019/0154667 A1
(43) Pub. Date: May 23, 2019

(54) ORGANOTYPIC PIG RETINA CULTURE SYSTEM

(71) Applicant: Adverum Biotechnologies, Inc., Menlo Park, CA (US)

(72) Inventors: Annahita KERAVALA, Palo Alto, CA (US); Diana CEPEDA, Menlo Park, CA (US)

(21) Appl. No.: 16/098,354

(22) PCT Filed: May 2, 2017
(86) PCT No.: PCT/US2017/030636

§ 371 Date: Nov. 1, 2018

Related U.S. Application Data
(60) Provisional application No. 62/331,295, filed on May 3, 2016.

(51) Int. Cl. G01N 33/50; C12N 5/079

(52) U.S. Cl. CPC G01N 33/5044; C12N 2510/00; C12N 2500/32; C12N 5/0621

(57) ABSTRACT

The present invention provides organotypic pig and non-human primate retina culture systems, and methods for evaluating or characterizing candidate therapeutic agents e.g. AAV-mediated gene vectors, using the same.

### CROSS REFERENCE TO RELATED APPLICATIONS

This application claims the benefit of U.S. Provisional Application No. 62/331,295 filed May 3, 2016, the disclosure of which is hereby incorporated by reference herein in its entirety.

### FIELD OF THE INVENTION

This invention relates to organotypic porcine and non-human primate retinal explant culture systems, and methods for evaluating or characterizing candidate therapeutic agents e.g. AAV-mediated gene vectors.

### BACKGROUND

A major challenge for the development of novel therapeutics is the limited availability of relevant model systems in which knowledge gained from high-throughput, genomic, and proteomic approaches can be integrated to study function. Animal models are still the main choice for such studies, but over the past few years powerful new in vitro systems have begun to emerge as useful tools to study function. While gene therapy studies utilizing in vivo animal models provide critical information regarding vector specificity, therapeutic efficacy, and safety, the long durations to observe transgene expression, high degree of variability between animals, and reduced control of experimental conditions often requires large sample sizes and expense.

Organotypic culture involves the ex vivo maintenance of an organ or tissue, or parts thereof, as an in vitro model that recapitulates the in vivo cellular architecture of a tissue. Therefore, organotypic culture methods allow investigators to observe and manipulate the behavior of complete tissues in a highly controlled in vitro setting while maintaining cells in situ with at least some semblance of physiological intercellular processes and communications. However, organotypic culture can have technical difficulties, such as the amount of time the cultured tissue or cells can remain viable in culture. Further, selecting and optimizing a system that best models a human organ or tissue can be difficult. Many existing protocols for organotypic culture rely on tissue harvested from embryonic, fetal, or juvenile animals, which often do not yet have established anatomy or intercellular connections that are seen in the adult.

Organotypic culture could benefit efforts to characterize and evaluate novel therapeutics that target the retina, however, optimal organotypic retina culture systems that accurately model the human retina are lacking. There is a clear need in the art for organotypic retina culture systems that accurately model the human retina with long-lasting anatomical and cellular viability.

### BRIEF SUMMARY OF THE INVENTION

The present invention relates generally to an organotypic culture system that contains a porcine or non-human primate retinal explant comprising live cells and a cell culture medium. The organotypic retina culture system is suitable for screening candidate agents to determine their suitability for use in gene therapy treatments.

Particular embodiments of the present invention are directed to an organotypic culture system comprising a retinal explant comprising live cells and a cell culture medium; wherein the retinal explant is obtained from an eye from a pig or a non-human primate.

In some embodiments, the live cells are viable in the organotypic culture system for about two to about four weeks. In certain embodiments, the tissue architecture of the retinal explant is preserved for about two to about four weeks. In particular embodiments the live cells comprise a cell that expresses beta-III-tubulin, a cell that expresses Chx10, a cell that expresses rhodopsin, and/or a cell that expresses L/M opsin. In some embodiments, the live cells comprise: ganglion cells, bipolar cells, cones, rods, retinal pigment epithelial cells, amacrine cells, horizontal cells, astrocytes, and/or Müller cells.

In particular embodiments, the porcine retinal explant comprises an a nerve fiber layer, a ganglion cell layer, an inner plexiform layer, an inner nuclear layer, an outer plexiform layer, an outer nuclear layer, an external limiting membrane, and/or a layer of rods and cones. In some embodiments, the retinal explant comprises all of: the nerve fiber layer, the ganglion cell layer, the inner plexiform layer, the inner nuclear layer, the outer plexiform layer, the outer nuclear layer, the external limiting membrane, and the layer of rods and cones.

In certain embodiments, the non-human primate retinal explant comprises an inner a nerve fiber layer, a ganglion cell layer, an inner plexiform layer, an inner nuclear layer, an outer plexiform layer, an outer nuclear layer, an external limiting membrane, a layer of rods and cones and/or the retinal pigment epithelium. In some embodiments, the retinal explant comprises all of: the nerve fiber layer, the ganglion cell layer, the inner plexiform layer, the inner nuclear layer, the outer plexiform layer, the outer nuclear layer, the external limiting membrane, the layer of rods and cones, and/or the retinal pigment epithelium. In some embodiments, the retinal explant has a diameter of between about 4 mm to about 16 mm.

In particular embodiments, the retinal explant is obtained from an adult pig or an adult non-human primate. In some embodiments, the retinal explant is obtained from an adult pig that is about six to about seven months old. In certain embodiments, the porcine retinal explant is obtained from an American Yorkshire pig.

In particular embodiments, the organotypic culture system comprises a transwell containing the retinal explant, and wherein the transwell is inserted into a well containing the cell culture medium. In some embodiments, the transwell comprises a top compartment, wherein the top compartment is composed of a circular wall and a porous membrane that forms the base of the top compartment, wherein the porous membrane comprises a top surface and a bottom surface, wherein the retinal explant is contained in the top compartment and contacts the top surface of the membrane; and wherein the bottom surface of the membrane contacts the cell culture medium. In certain embodiments, the retinal explant comprises a surface comprising an exposed photoreceptor layer or an exposed retinal pigment epithelium and a surface comprising an exposed nerve fiber layer, and wherein the face comprising the exposed photoreceptor cell layer or exposed retinal pigment epithelium contacts the top surface of the membrane. In some embodiments, the surface comprising the exposed photoreceptor layer or the exposed retinal pigment epithelium is exposed to the cell culture medium, and the surface comprising the exposed nerve fiber layer is exposed to atmosphere.

In certain embodiments, the organotypic culture system comprises a cell culture medium that comprises Neurobasal A. In some embodiments, the cell culture medium comprises an L-glutamine supplement, a neuron supplement, and at least one antibiotic. In particular embodiments, the L-glutamine supplement is GlutaMAX, the neuron supplement is B-27, and the at least one antibiotic is a combination of penicillin, streptomycin, amphotericin B, and a growth factor.

In some embodiments, the retinal explant comprises a plurality of the live cells that comprise an exogenous polynucleotide. In particular embodiments, the exogenous polynucleotide is an expression vector, an mRNA, an expression cassette, or an oligonucleotide. In some embodiments, the expression vector is a virus, optionally an adeno-associated virus (AAV), an adenovirus, or a lentivirus.

Particular embodiments of the present invention are drawn to a method of determining expression of an exogenous polynucleotide in a retinal explant, comprising: (A) isolating the retinal explant from an eye of a pig or a non-human primate, wherein the portion of the retinal explant comprises live cells; (B) contacting the retinal explant with the exogenous polynucleotide; (C) incubating the retinal explant in an organotypic culture system for a period of time; and (D) determining the expression of the exogenous polynucleotide in the retinal explant; thereby determining the expression of the exogenous polynucleotide; wherein the retinal explant is a porcine retinal explant or a non-human primate retinal explant. In some embodiments, the exogenous polynucleotide is an expression vector, an mRNA, an expression cassette, or an oligonucleotide. In particular embodiments, the expression vector is a virus, optionally an adeno-associated virus (AAV).

In particular embodiments, the of step isolating the retinal explant from an eye of a pig or a non-human primate, wherein the portion of the retinal explant comprises live cells, comprises the steps of (i) obtaining the eye from the pig or non-human primate; (ii) isolating a retina from the eye; and (iii) removing a portion from the retina; thereby isolating the retinal explant from the eye. In some embodiments, the step of obtaining the eye from the pig or non-human primate, comprises enucleating the eye from the pig or non-human primate within two hours postmortem and placing the eye in cold phosphate buffered saline (PBS). In certain embodiments, the pig or non-human primate is an adult animal. In particular embodiments, the adult pig is about six to about seven months old. In some embodiments, the pig is an American Yorkshire pig.

In particular embodiments, the step of isolating a retina from the eye, comprises the steps of: (i) cutting the eye along the corneal limbus of the eye into an anterior portion and a posterior portion, wherein the posterior portion comprises an optic disc and the retina; (ii) placing the posterior portion into a dissection medium; (iii) removing the retina from the posterior portion; thereby isolating the retina from the eye. In some embodiments, the extraneous tissue is removed from the eye prior to performing the step of cutting the eye along the corneal limbus of the eye. In some embodiments, the dissection medium is a CO2-independent medium.

In some embodiments, the optic disc of the posterior portion is severed prior to performing the step of removing the retina from the posterior portion. In certain embodiments, the step of removing the retina from the posterior portion comprises cutting the retina with a circular biopsy punch, thereby obtaining the retinal explant. In some embodiments, the retinal explant has a diameter of about 4 mm to about 6 mm.

In particular embodiments, the porcine retinal explant comprises a nerve fiber layer, a ganglion cell layer, an inner plexiform layer, an inner nuclear layer, an outer plexiform layer, an outer nuclear layer, an external limiting membrane, and/or a layer of rods and cones. In certain embodiments, the porcine retinal explant comprises all of: the nerve fiber layer, the ganglion cell layer, the inner plexiform layer, the inner nuclear layer, the outer plexiform layer, the outer nuclear layer, the external limiting membrane, and the layer of rods and cones.

In some embodiments, the non-human primate retinal explant comprises wherein the non-human primate retinal explant comprises an inner a nerve fiber layer, a ganglion cell layer, an inner plexiform layer, an inner nuclear layer, an outer plexiform layer, an outer nuclear layer, an external limiting membrane, a layer of rods and cones and/or the retinal pigment epithelium. In certain embodiments, the non-human primate retinal explant wherein the retinal explant comprises all of: the nerve fiber layer, the ganglion cell layer, the inner plexiform layer, the inner nuclear layer, the outer plexiform layer, the outer nuclear layer, the external limiting membrane, the layer of rods and cones, and the retinal pigment epithelium.

In some embodiments, the step of contacting the retinal explant with the exogenous polynucleotide comprises the steps of; (i) placing the retinal explant in a cell culture dish, in a well of a cell culture plate, or in a transwell that is contained in a well of a cell culture plate; and (ii) contacting the porcine retinal explant with a solution comprising the exogenous polynucleotide; and thereby contacting the portion of the retina to a exogenous polynucleotide. In particular embodiments, the transwell comprises a top compartment, wherein the top compartment is composed of a circular wall and a porous membrane that forms the base of the top compartment, wherein the porous membrane comprises a top surface and a bottom surface, and wherein the porcine retinal explant is placed in the top compartment of the transwell. In some embodiments, the retinal explant comprises a surface comprising an exposed photoreceptor layer or an exposed retinal pigment epithelium and a surface comprising an exposed nerve fiber layer, and the face comprising the exposed photoreceptor cell layer or exposed retinal pigment epithelium is placed in the top compartment of the transwell and contacts the top surface of the membrane. In some embodiments, the face comprising the exposed photoreceptor layer or the exposed retinal pigment epithelium is exposed to the cell culture medium, and the surface comprising the exposed nerve fiber layer is exposed to atmosphere.

In some embodiments, the step of contacting the porcine retinal explant with a solution comprising the exogenous polynucleotide is performed by placing the solution into the top compartment of the transwell, thereby contacting the exogenous polynucleotide to the porcine retinal explant.

In certain embodiments, the exogenous polynucleotide is a virus and the porcine retinal explant is contacted with virus in an amount of about 1×103 to about 1×105 multiplicity of infection (MOI). In some embodiments, the amount is about 2×104 MOI. In certain embodiments, the exogenous polynucleotide is a virus, and wherein the porcine retinal explant is contacted with the virus at a concentration of about 1×104 to about 1×1012 polynucleotide genomes per microliter. In some embodiments, the concentration is about 2×10⁸ vector genomes per microliter. In certain embodiments, the exogenous polynucleotide contacts the porcine retinal explant for about 15 minutes to about 12 hours. In particular embodiments, the exogenous polynucleotide contacts the porcine retinal explant for about 2 hours.

In particular embodiments, the step of incubating the retinal explant in an organotypic culture system for a period of time comprises the steps of: (i) placing the retinal explant in a cell culture dish, a well of a cell culture plate, or a transwell that is contained in a well of a cell culture plate; and (ii) adding an organotypic culture medium to the cell culture dish, the well of the cell culture plate, or the transwell. In certain embodiments, the transwell comprises a top compartment, wherein the top compartment is composed of a circular wall and a porous membrane that forms the base of the top compartment, wherein the porous membrane comprises a top surface and a bottom surface, and wherein the retinal explant is placed in the top compartment of the transwell, and wherein the step of adding an organotypic culture medium comprises adding an organotypic culture medium to the well containing the transwell, wherein the organotypic culture medium is added to the height of the membrane of the transwell so that the organotypic culture medium contacts the bottom surface of the porous membrane. In some embodiments, the retinal explant comprises a surface comprising an exposed photoreceptor layer or an exposed retinal pigment epithelium and a surface comprising an exposed nerve fiber layer, and wherein the surface comprising the exposed photoreceptor cell layer or exposed retinal pigment epithelium is placed in the top compartment of the transwell and contacts the top surface of the membrane, and wherein the face comprising the exposed photoreceptor layer or the exposed retinal pigment epithelium is exposed to the cell culture medium, and the surface comprising the exposed nerve fiber layer is exposed to atmosphere.

In some embodiments, the organotypic culture medium is added into a well containing the transwell, wherein the organotypic culture medium is added to the height of the membrane of the transwell so that the organotypic culture medium contacts the bottom surface of the porous membrane, and so that the organotypic culture medium contacts the porcine retinal explant at the surface exposing the retinal pigment epithelium and does not contact the porcine retinal explant at a surface exposing the inner limiting membrane. In certain embodiments, the cell culture medium that comprises Neurobasal A. In some embodiments, the cell culture medium comprises an L-glutamine supplement, a neuron supplement, and at least one antibiotic. In particular embodiments, the L-glutamine supplement is GlutaMAX, the neuron supplement is B-27, and the at least one antibiotic is a combination of penicillin, streptomycin, amphotericin B, and a growth factor.

In some embodiments, the retinal explant is incubated in the organotypic culture system for a period of time is between about 3 days and about 42 days. In particular embodiments, the period of time is between about 14 days and 28 days. In some embodiments, at least a portion of cells of the porcine retinal explant are viable during the period of time. In particular embodiments, the exogenous polynucleotide encodes a polypeptide.

In particular embodiments, the step of determining the expression of the exogenous polynucleotide in the retinal explant comprises detecting the polypeptide. In some embodiments, the polypeptide is a therapeutic polypeptide. In certain embodiments, the polypeptide is a fluorescent polypeptide. In particular embodiments, the fluorescent polypeptide comprises green fluorescent protein (GFP). In some embodiments, the polypeptide is a secreted polypeptide. In certain embodiments, the secreted polypeptide is VEGFR-1 or another protein that binds to VEGF.

In some embodiments, the polypeptide is detected by live cell imaging, immunofluorescence, western blotting, and/or ELISA. In particular embodiments, detecting the polypeptide further comprises labeling a population of cells of the porcine retinal explant. In certain embodiments, labeling the population of cells of the porcine retinal explant comprises contacting the population with an antibody that recognizes a marker of the population of cells, and detecting the antibody with immunofluorescence. In some embodiments, the population of cells is selected from the group consisting of: ganglion cells, bipolar cells, cones, rods, retinal pigment epithelial cells, amacrine cells, horizontal cells, astrocytes, and Müller cells.

### BRIEF DESCRIPTION OF THE DRAWINGS

FIG. 1A-FIG. 1C show data demonstrating the viability of porcine retinal explants ex vivo. FIG. 1A shows glucose-6-phosphate dehydrogenase release from dying cells in four different retinal explants during the first week in culture. FIG. 1B shows glucose-6-phosphate dehydrogenase release from dying cells in eleven different retinal explants during over a period of 3.5 weeks in culture. The bottom line at day 7 graphs glucose-6-phosphate dehydrogenase release from cultured HEK293 cells over the same time period. FIG. 1C shows TUNEL staining of transverse sections of retinal explants over a period of weeks. DAPI staining is observed for Day 1 through Week 4, wherein ApopTag staining is seen below the DAPI staining for Week 1 and Week 2.

FIG. 2A-FIG. 2C show immunofluorescence staining of transverse sections of explants for retinal cell markers. FIG. 2A shows staining for bipolar cells and cone cells, FIG. 2B shows staining for Muller glial cells, bipolar cells, and rod cells. FIG. 2C shows staining for renal ganglion cells (RGC), and rod cells. Antibodies against the following proteins were used: CHX10 for biopolar cells, L/M opsin for cone cells, GFAP for Muller glia, Tujl/β-tubulin 3 for RGC, and rhodopsin for rod cells. All tissue sections were counterstained with DAPI for visualization of nuclei.

FIG. 3A-FIG. 3B show protein expression in AAV2.7m8-transduced explants over time. FIG. 3A shows live imaging of explants transduced with an AAV2.7m8 vector expressing GFP. FIG. 3B shows quantification (μg/mL) of secreted protein (sProtein) in supernatants from explants transduced with an AAV2.7m8 vector.

FIG. 4. A-FIG. 4B show GFP expression from different AAV variants. FIG. 4A shows live imaging of GFP in explants at 1 and 2 weeks prost-transduction with the indicated AAV variants. All images were acquired at the same exposure. FIG. 4B shows immunofluorescence of pig retinal explant tissue sections at 2 weeks post-transduction co-labeled with GFP and retinal markers rhodopsin, GFAP (for rod and Mueller glial cells, respectively), and DAPI (for nuclei).

FIG. 5A-FIG. 5C show immunofluorescence staining of GFP expression 2 weeks after transduction with CAG (FIG. 5A), CMV (FIG. 5B), or MNTC (FIG. 5C) promoter-driven cassettes. GFP was co-labeled with Rhodopsin staining of rod cells and DAPI staining for nuclei.

FIG. 6 shows quantification of secreted sVEGFR1 in supernatants from porcine retinal explants transduced with different AAV vectors.

### DETAILED DESCRIPTION OF THE INVENTION

Unless otherwise defined herein, scientific and technical terms used in this application have the meanings commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The term “organotypic culture” is meant that cultured cells are associated in a way that closely replicates the biochemical and physiological properties of the organ from which the cells are derived. This may be achieved by culturing an intact portion of an organ or tissue.

The term “retina” as used herein refers to the layer at the back of the eyeball containing cells that are sensitive to light and that trigger nerve impulses that pass via the optic nerve to the brain, where a visual image is formed.

The term “enucleation” as used herein refers to a general surgical technique of removing a mass without cutting or dissecting it.

“Ganglion cell” or “retinal ganglion cell” is a type of neuron located near the inner surface “ganglion cell layer” of the retina of the eye. It receives visual information from photoreceptors via two intermediate neuron types: bipolar cells and amacrine cells.

A “bipolar cell” is a neuron of the retina that exists between photoreceptors (rod cells and cone cells) and ganglion cells. Bipolar cells act, directly or indirectly, to transmit signals from the photoreceptors to the ganglion cells.

“Cones” or “cone cells” are a type of photoreceptor cells in the retina of the eye. They are responsible for color vision and function best in relatively bright light. In humans, cone cells are densely packed in the fovea centralis, a 0.3 mm diameter rod-free area with very thin, densely packed cones which quickly reduce in number towards the periphery of the retina.

“Rods” or “rod cells” are photoreceptor cells in the retina of the eye that can function in less intense light than cone cells. Rods are concentrated at the outer edges of the retina and are used in peripheral vision. On average, there are approximately 90 million rod cells in the human retina. More sensitive than cone cells, rod cells are almost entirely responsible for night vision. However, because they have only one type of light-sensitive pigment, rather than the three types that human cone cells have, rods have little, if any, role in color vision

“Retinal pigment epithelial cells” refer to cells of the retinal pigment epithelium (RPE), the pigmented cell layer adjacent to the neurosensory retina.

“Amacrine cells” are inhibitory interneurons in the retina which project their dendritic arbors to the inner plexiform layer (IPL), where they synapse with retinal ganglion cells and/or bipolar cells.

“Horizontal cells” are the laterally interconnecting neurons having cell bodies in the inner nuclear layer of the retina. These cells integrate and regulate the input from multiple photoreceptor cells. Among their functions, horizontal cells are responsible for allowing eyes to adjust to see well under both bright and dim light conditions.

“Müller cells” are the most common retinal glial cell and span across the distinct anatomical layers of the retina.

The term “explant” as used herein refers to a portion of an organ or tissue, e.g. retinal tissue, that is transferred from an organism and is placed into a culture system.

The “corneal limbus” is the border of the cornea and the sclera (the white of the eye).

The “optic disc” is the raised disk on the retina at the point of entry of the optic nerve, lacking visual receptors and so creating a blind spot.

The “inner limiting membrane” is the boundary between the retina and the vitreous body, formed by astrocytes and the end feet of Müller cells. It is separated from the vitreous humor by a basal lamina.

The “nerve fiber layer,” also referred to as the stratum opticum or retinal nerve fiber layer (RNFL), is formed by the expansion of the fibers of the optic nerve; it is thickest near the porus opticus, gradually diminishing toward the ora serrata.

The “ganglion cell layer” is a layer of the retina that consists of retinal ganglion cells and displaced amacrine cells.

“Inner plexiform layer” refers to an area of the retina that is made up of a dense reticulum of fibrils formed by interlaced dendrites of retinal ganglion cells and cells of the inner nuclear layer.

The “inner nuclear layer” or “layer of inner granules” of the retina is made up of a number of closely packed bipolar cells, horizontal cells, and amacrine cells.

The “outer plexiform layer” is a layer of neuronal synapses in the retina of the eye. It consists of a dense network of synapses between dendrites of horizontal cells from the inner nuclear layer, and photoreceptor cell inner segments from the outer nuclear layer.

The “outer nuclear layer” of the retina contains several strata of rod and cone nuclear bodies, i.e., granules.

The “external limiting membrane” of the retina is situated at the base of rods and cones of rod cells and cone cells.

The “layer of rods and cones” of the retina, also known as Jacob's membrane, is composed of the rods and cones of the rod cells and cone cells.

The “retinal pigment epithelium” is the pigmented cell layer just outside the neurosensory retina that nourishes retinal visual cells, and is firmly attached to the underlying choroid and overlying retinal visual cells.

As used herein, “transwell” or “transwell insert” refers to commercially available plastic inserts for culture dishes or for wells of multi-well plates. The transwells possess a porous membrane, as typified by Corning TRANS WELLS®. When placed in the well of a multi-well tissue culture plate these inserts create a two-chamber system separated by the porous membrane.

### Organotypic Culture System

Particular embodiments of the present invention relate to an organotypic retinal culture system. The organotypic retinal culture system generally includes a porcine or non-human primate retinal explant containing live cells and a cell culture medium. In certain embodiments, the retinal explant is a porcine retinal explant. The organotypic retinal culture system may further include any of the elements described herein. Certain embodiments contemplate that the organotypic culture system of the present invention is useful for, inter alia, determining the expression of an exogenous polynucleotide in retinal tissue.

In certain embodiments, organotypic retinal culture systems comprise a retinal explant that comprises live cells and is contained in the top compartment of a transwell, wherein the retinal explant has intact distinct morphological layers that include the nerve fiber layer, the ganglion cell layer, the inner plexiform layer, the inner nuclear layer, the outer plexiform layer, the outer nuclear layer, the external limiting membrane, and the layer of rods and cones (i.e. the photoreceptor layer. In particular embodiments, the surface of the retinal explant with exposed photoreceptor layer is placed face down on the transwell membrane that forms the bottom of the top compartment. The well that houses the transwell contains a cell culture medium comprising Neurobasal A supplemented with GlutaMAX, B27, and 100 units/mL of penicillin, 100 μg/mL of streptomycin, and 0.25 μg/mL Amphotericin B. The cell culture medium is filled to the height of the transwell membrane, so that the surface of the explant that exposes the photoreceptor layer contacts the medium, but the opposite surface does not. Some embodiments contemplate that the live cells in the retinal explant remain viable for at least two to four weeks. In some embodiments, the retinal explant is a mammalian retinal explant, e.g., a porcine retinal explant, or a non-human primate retinal explant. Illustrative embodiments described herein relate to porcine retinal explants, but it is understood that other mammalian retinal explants and non-human primate retinal explants may be substituted for the porcine retinal explant in any embodiment.

### Retina

In a particular embodiment, the present invention provides a method of producing an organotypic retinal culture from a pig, another mammal, or non-human primate retinal explant, the method comprising culturing a porcine, mammalian, or non-human primate retinal explant.

The retina is a light-sensitive layer of tissue located on the inner surface of the eye. The optics of the eye project an image of the visual world on the retina through the cornea and lens, which serves much the same function as the film in a camera. Light striking the retina initiates a cascade of chemical and electrical events that ultimately trigger nerve impulses. These are sent to various visual centers of the brain through the fibers of the optic nerve.

In vertebrate embryonic development, the retina and the optic nerve originate as outgrowths of the developing brain, so the retina is considered part of the central nervous system (CNS).

The retina is a layered structure with several layers of neurons interconnected by synapses. The only neurons that are directly sensitive to light are the photoreceptor cells which consist of mainly of two types: the rods and cones. Rods provide black-and-white vision, while cones support perception of color. A third, much rarer type of photoreceptor, the intrinsically photosensitive ganglion cell, is important for reflexive responses to bright daylight.

The human retina has ten distinct layers. From closest to farthest from the vitreous body—that is, from closest to the front exterior of the head towards the interior and back of the head: an inner limiting membrane, a nerve fiber layer, a ganglion cell layer, an inner plexiform layer, an inner nuclear layer, an outer plexiform layer, an outer nuclear layer, an external limiting membrane, a layer of rods and cones, and/or a retinal pigment epithelium.

In adult humans, the entire retina contains about 7 million cones and 75 to 150 million rods. The optic disc, a part of the retina sometimes called “the blind spot” because it lacks photoreceptors, is located at the optic papilla, a nasal zone where the optic-nerve fibers leave the eye.

### Pig Retina

Without being bound by theory, particular embodiments contemplate that pig retina is particularly useful for modeling the human retina because the porcine retina and the human retina contain similar morphology. The pig eye and the pig retina shares many similarities with that of the human. The pig eye is slightly smaller than the human eye, but has some unique advantages that directly apply to the human eye. Pigs have a scleral thickness that is very similar to humans. The pig eye also has a holangiotic retina with a vascular pattern very similar to humans, a cone-dense region simulating a human macula, and retinal pigment epithelium with choroid very similar to humans. The porcine retina shares a greater similarity to the human retina than that of other large mammals such as the dog, goat, cow, or ox (Discussed in Prince, J. H., Diesem, C. D., Eglitis, I., Ruskell, G. L., 1960. The pig. In: Thomas, C. C. (Ed.), Anatomy and Histology of the Eye and Orbit in Domestic Animals. Springfield, Ill., pp. 210-230.; which is herein incorporated by reference in its entirety.) However, a difficulty with the use of pigs to model the human eye has been their expense, size, and difficulty to handle in laboratory settings as compared to smaller lab animals.

In certain embodiments, the organotypic culture system contains a porcine retinal explant that maintains an intact morphology for a period of time. By “intact morphology” it is meant that the portion of pig retina maintains its layered morphology with distinct organization and organization of specific cell types. In certain embodiments, the intact morphology of the porcine retinal explant substantially persists in the organotypic culture system for at least about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 35 days, or at least about 42 days.

In certain embodiments, the porcine retinal explant comprises one or more distinct morphological layers. As with human retina, the distinct morphological layers of pig retina are the inner limiting membrane, the nerve fiber layer, the ganglion cell layer, the inner plexiform layer, the inner nuclear layer, the outer plexiform layer, the outer nuclear layer, the external limiting membrane, the layer of rods and cones, and the retinal pigment epithelium. In some embodiments, the porcine retinal explant contains two, three, four, five, six, seven, eight, nine, or all ten of the distinct morphological layers. In particular embodiments, the porcine retinal explant comprises the nerve fiber layer, the ganglion cell layer, the inner plexiform layer, the inner nuclear layer, the outer plexiform layer, the outer nuclear layer, the external limiting membrane, and the layer of rods and cones. In some embodiments, at least six, at least seven, or at least eight distinct morphological layers are maintained in the porcine retinal explant under the organotypic culture system at least about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 35 days, or at least about 42 days. In particular embodiments, the ten distinct morphological layers are maintained in the organic culture system for at least about 14 days. In certain embodiments, the ten distinct morphological layers are maintained in the organic culture system for at least about 28 days.

By maintaining distinct morphological layers, it is meant that the porcine retinal explant contains viable (i.e. live) cells within the distinct morphological layer for a period of time, and that the viable cells maintain their morphology for the period of time, and that each layer remains distinct from the adjacent layers with respect to cell identity and cell shape.

In some embodiments, the porcine retinal explant comprises some or all of the distinct morphological layers and is circular in shape. In certain embodiments, the porcine retinal explant has a diameter of about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm. In particular embodiments, the porcine retinal explant has a diameter of between about 100 μm and about 20 mm, between about 500 μm and about 16 mm, between about 1 mm and about 16 mm, between about 2 mm and about 8 mm, or between about 4 mm and about 6 mm. In certain embodiments, the non-human primate retinal explant is about 4 mm to about 16 mm in diameter.

In particular embodiments, at least a fraction of the cells in the porcine retinal explant are viable in the organotypic culture system for a period of time. In certain embodiments, at least 5%, at least 10%, at least 15%, at least 20%, at least 25%, at least 30%, at least 35%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99% of the cells remain viable in the organotypic cell culture system for a period of time. In particular embodiments, the period of time is at least about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 35 days, or at least about 42 days.

In certain embodiments, the porcine retinal explant contains live cells that include at least one cell type, including but not limited to, ganglion cells, bipolar cells, cones, rods, retinal pigment epithelial cells, amacrine cells, horizontal cells, astrocytes, and/or Müller cells. In some embodiments, the live cells comprise a cell that expresses beta-III-tubulin, which is expressed by neurons including photoreceptors, bipolar cells, ganglion cells, horizontal cells, and amacrine cells; a cell expressing Chx10, which is expressed by bipolar cells; a cell that expresses rhodopsin, which is expressed by rod cells; and/or a cell that expresses L/M opsin, which is expressed by cone cells.

### Pig

Pigs serve as animal models to model organ systems including the cardiovascular system, lung, gastrointestinal system, liver, pancreas, kidney, lower urinary system, skin, reproductive system, endocrine system, lymphatic and hematopoietic systems, central nervous system. Thus, in some embodiments, a pig eye is obtained postmortem from a pig that was killed for a research purpose other than to study the retina or generate an organotypic retinal culture.

In some embodiments, the porcine retinal explant is obtained from an eye of a miniature pig breed. In some instances, miniature pig breeds have been generated for research purposes. The differences between domestic farm breeds and miniature breeds are related to their growth rate and size at sexual maturity, rather than actual anatomic differences in organs and structures. Thus, when different breeds are age matched, the organ sizes will reflect the increased size of domestic breeds compared with miniature breeds; however, the physiologic function should be the same. Conversely, when animals are weight matched, the sizes will be similar for organs and structures; however, the physiologic function will be related to the relative maturity of the animals.

The most common miniature breeds available in the United States are the Hanford, Yucatan, Yucatan micro, Sinclair, and Göttingen (from largest to smallest). These miniature breeds weigh 0.5-1 kg at birth and grow to 17-20 kg for the Hanford and 7-9 kg for the Göttingen in 4 months. Sexual maturity for all breeds occurs between 4 and 6 months of age. In certain embodiments, the portion of the pig retina is obtained from a Yucatan, a Yucatan micro, a Sinclair, or a Göttingen pig.

In some embodiments, the porcine retinal explant is obtained from a domestic pig. These breeds are generally larger than the miniature pigs, and can be found in farm settings as well as in research settings. Suitable pig breeds for obtaining a retina include, but are not limited to, American Landrace, American Yorkshire, Berkshire, Mulefoot, Tamworth, Large Black, Hereford, Poland China, Chester White, Landrace, Hampshire, Duroc, and Yorkshire. In particular embodiments, the porcine retinal explant is obtained from an eye of an American Yorkshire pig.

In particular embodiments, the organotypic culture system contains porcine retinal explant that is obtained from an eye of an adult pig. In some embodiments, the adult pig is at least about 5 months, about 6 months, about 7 months, about 8 months, about 9 months, about 10 months, about 11 months, about 12 months, about 18 months, about 24 months old when the eye is removed.

In certain embodiments, the organotypic culture system contains a pig retina that is obtained from a pig eye ball that is removed from the pig postmortem. In some embodiments, the pig eye is removed from the pig within about 24 hours, within about 8 hours, within about 6 hours, within about 5 hours, within about 4 hours, within about 3 hours, within about 2 hours, or within about 1 hour postmortem. In particular embodiments, the pig eye is removed within about 180 minutes, about 150 minutes, about 120 minutes, about 90 minutes, about 60 minutes, about 45 minutes, about 30 minutes, or less than 30 minutes postmortem.

### Non-Human Primate Retina

Without being bound by theory, certain embodiments contemplate that non-human primate retina is particularly useful for modeling the human retina because the non-human primate retina and the human retina contain similar morphology.

In certain embodiments, the organotypic culture system contains a non-human retinal explant that maintains an intact morphology for a period of time. In certain embodiments, the intact morphology of the non-human primate retinal explant substantially persists in the organotypic culture system for at least about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 35 days, or at least about 42 days.

In certain embodiments, the non-human retinal explant comprises one or more distinct morphological layers. As with human retina, the distinct morphological layers of the non-human primate retina are the inner limiting membrane, the nerve fiber layer, the ganglion cell layer, the inner plexiform layer, the inner nuclear layer, the outer plexiform layer, the outer nuclear layer, the external limiting membrane, the layer of rods and cones, and the retinal pigment epithelium. In some embodiments, the non-human primate retinal explant contains two, three, four, five, six, seven, eight, nine, or all ten of the distinct morphological layers. In various embodiments, the non-human primate retinal explant contains the nerve fiber layer, the ganglion cell layer, the inner plexiform layer, the inner nuclear layer, the outer plexiform layer, the outer nuclear layer, the external limiting membrane, the layer of rods and cones, and the retinal pigment epithelium. In some embodiments, at least one, at least two, at least three, at least four, at least five, at least six, at least seven, at least eight, or at least nine distinct morphological layers are maintained in the non-human retinal explant under the organotypic culture system at least about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 35 days, or at least about 42 days. In particular embodiments, the ten distinct morphological layers are maintained in the organic culture system for at least about 14 days. In certain embodiments, at least eight or nine distinct morphological layers are maintained in the organic culture system for at least about 28 days.

In some embodiments, the non-human primate retinal explant comprises some or all of the distinct morphological layers. In certain embodiments, the non-human primate retinal explant has a diameter of about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15, or about 16 mm. In particular embodiments, the non-human primate retinal explant has a diameter of between about 100 μm and about 20 mm, between about 500 μm and about 16 mm, between about 1 mm and about 16 mm, between about 2 mm and about 8 mm, or between about 4 mm and about 6 mm. In certain embodiments, the non-human primate retinal explant is about 4 mm to about 16 mm in diameter.

In particular embodiments, at least a fraction of the cells in the non-human primate retinal explant are viable in the organotypic culture system for a period of time. In certain embodiments, at least 5%, at least 10%, at least 15%, at least 20%, at least 25%, at least 30%, at least 35%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99% of the cells remain viable in the organotypic cell culture system for a period of time. In particular embodiments, the period of time is at least about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 35 days, or at least about 42 days.

In certain embodiments, the non-human primate retinal explant contains live cells that include at least one cell type, including but not limited to, ganglion cells, bipolar cells, cones, rods, retinal pigment epithelial cells, amacrine cells, horizontal cells, astrocytes, and/or Müller cells. In some embodiments, the live cells comprise a cell that expresses beta-III-tubulin, which is expressed by neurons including photoreceptors, bipolar cells, ganglion cells, horizontal cells, and amacrine cells; a cell expressing Chx10, which is expressed by bipolar cells; a cell that expresses rhodopsin, which is expressed by rod cells; and/or a cell that expresses L opsin, M opsin, or S opsin, which are expressed by cone cells.

### Non-Human Primate

Non-human primates are used in research investigating neurobiology, behavior, cognition, reproduction, genetics, xenotransplantation, drug abuse, diseases such as neurodegenerative diseases (e.g. Parkinson's disease,) infectious diseases (e.g. HIV, malaria, and respiratory viruses) and also in vaccine and drug testing. In certain embodiments, the non-human primate is an ape, monkey, gorilla, chimp, baboon, orangutan, or lutung, or any other described herein. Most non-human primates used in research are macaques, such as Rhesus macaques and cynomolgous macaques. In addition, marmosets, tamarins, spider monkeys, owl monkeys, vervet monkeys, squirrel monkeys, and baboons are also used research.

In some embodiments, the retinal explant is obtained from an eye of a non-human primate. In certain embodiments, the retinal explant is obtained from a non-human primate. In particular embodiments, the retinal explant is obtained from a non-human primate that is a macaque (e.g. a Rhesus macaque or a cynomolgous macaque), a marmoset, a tamarin, a spider monkey, an owl monkey, a vervet monkey, a squirrel monkey, a baboon, or an orangatang. In particular embodiments, the organotypic culture system contains non-human primate retinal explant that is obtained from an eye of an adult non-human primate.

In certain embodiments, the organotypic culture system contains a non-human primate retina that is obtained from a non-human primate eye that is removed from the primate postmortem. In some embodiments, the eye is removed from the non-human primate within about 24 hours, within about 8 hours, within about 6 hours, within about 5 hours, within about 4 hours, within about 3 hours, within about 2 hours, or within about 1 hour postmortem. In particular embodiments, the non-human primate eye is removed within about 180 minutes, about 150 minutes, about 120 minutes, about 90 minutes, about 60 minutes, about 45 minutes, about 30 minutes, or less than 30 minutes postmortem.

### Culture

Particular embodiments of the organotypic culture system comprises a porcine or non-human primate retinal explant cultured in a cell culture dish, a well of a cell culture plate, or in a transwell placed in a cell culture dish or well of a cell culture plate.

In certain embodiments, the organotypic culture system comprises a porcine or non-human primate retinal explant cultured in a cell culture dish. In some embodiments, the cell culture dish has a circular shape. In particular embodiments, the retinal explant is cultured in a cell culture dish that has a diameter of 35 mm, 60 mm, 100 mm, 120 mm, 150 mm, or 200 mm.

In some embodiments, the organotypic culture system comprises a porcine or non-human primate retinal explant that is cultured in a well of a cell culture plate. In particular embodiments, a porcine or non-human primate retinal explant is placed in the well of a single well plate, a 2 well plate, a 6 well plate, a 12 well plate, a 24 well plate, a 48 well plate, or a 96 well plate.

In particular embodiments, the organotypic culture system comprises a porcine or non-human primate retinal explant that is cultured in a transwell. Transwells are cell culture inserts that can fit into a cell culture well. The transwell contains a top compartment, and the base of this compartment is a porous membrane. Cells or tissues can be cultured on the porous membrane in the top compartment. The well that the transwell insert is placed in can serve as a bottom compartment. A cell culture medium can be placed into the bottom compartment at a level so that the medium fills into the top compartment through the pores in the membrane. Alternatively, the medium can be filled in the bottom compartment to the height of membrane, so that cultured cells or tissue is contacted on its bottom surface with the medium, but is not completely submerged. Some embodiments contemplate that one advantage of a transwell insert is that it allows for a cultured tissue or cultured cells to be exposed to different conditions by filling the bottom compartment with one medium to the height of the membrane and a different medium, or no medium, to the top compartment, thus exposing different portions to the cultured tissue or cells to different conditions. Particular embodiments contemplate that an additional advantage is that the transwell system is that it can allow an experimenter to control which surface or portion of a tissue will contact a medium.

In some embodiments, the organotypic culture system comprises a porcine or non-human primate retinal explant that is cultured in a transwell insert in a 10 cm dish or in the well of a 6 well plate, a 12 well plate, a 24 well plate, a 48 well plate, or a 96 well plate. In particular embodiments, the organotypic culture system comprises a porcine or non-human primate retinal explant that is cultured in a transwell that is 1 mm, 2 mm, 3 mm, 4 mm, 5 mm, 6 mm, 7 mm, 8 mm, 9 mm, 10 mm, 11 mm, 12 mm, 15 mm, 18, mm, 20 mm, 24 mm, 30 mm, 35 mm, 40 mm, 45 mm, 50 mm, 60 mm, 70 mm, 80 mm, 90 mm, or 100 mm in diameter. In particular embodiments, the organotypic culture system comprises a porcine or non-human primate retinal explant that is cultured in the top compartment of a transwell that contains a porous membrane with 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.4 μm, 1.6 μm, 1.8 μm, 2 μm, 3 μm, or 4 μm pores.

In certain embodiments, the organotypic culture system comprises a porcine or non-human primate retinal explant that is cultured in a transwell so that the surface of the retinal explant with exposed retinal pigment epithelium or photoreceptor layer is placed in direct contact with the porous bottom surface of the transwell, and the surface of the retinal explant exposing the inner limiting membrane is exposed facing upwards. In this arrangement, the photoreceptor side of the retinal explant contacts the membrane of the transwell.

In some embodiments, the organotypic culture system comprises a porcine or non-human primate retinal explant that is cultured in a transwell insert, wherein the well that houses the transwell is filled with medium to the height of the membrane of the transwell, so that the surface of the porcine or non-human primate retinal explant that exposes retinal pigment epithelium layer or photoreceptor layer contacts the media through the pores in the membrane of the transwell, and the surface of the retinal explant exposing the nerve fiber layer or an adjacent layer is exposed to the atmosphere.

In some embodiments, the organic culture system comprises a porcine or non-human primate retinal explant that is cultured in the top compartment of a transwell, wherein the well that house the transwell contains feeder cells that are retinal pigment epithelial cells. In particular embodiments, the retinal pigment epithelial cells are cells from a stable cell line, for example but not limited to, hTERT RPE-1 cells or ARPE-19 cells. In some embodiments, the retinal pigment epithelial cells are primary cells.

### Culture Media

In some embodiments, the organotypic culture system comprises a porcine or non-human primate retinal explant cultured in a medium that is suitable for cell culture. In particular embodiments, the cell culture medium comprises a basal medium. In some embodiments, the basal medium is Neurobasal, Neurobasal A, DMEM, RPMI 1640, MEM, and/or IMDM basal medium. In particular embodiments, the porcine or non-human primate retinal explant is cultured in a culture medium suitable for cultured primary neurons, neuron like cells, and/or primary retinal cells. In certain embodiments, the medium is a serum free medium.

In some embodiments, the organotypic culture system comprises a porcine or non-human primate retinal explant that is cultured in a medium that contains a neuron cell culture supplement. In particular embodiments, the neuron cell culture supplement is B27, N2, or BIT9500. B27 is known in the art and contains amounts of bovine serum albumen (BSA), transferrin, insulin, progesterone, putrescine, sodium selenite, biotin, L-carnitine, corticosterone, ethanolamine, d(+)-galactose, reduced glutathione, linolenic acid, linoleic acid, retinyl acetate, selenium, T3 (triodo-1-thyronine), dl-alpha-tocolpherol, dl-alpha-tocopherol acetate, catalase, and superoxide dismutase. In certain embodiments, the organotypic culture system comprises a porcine or non-human primate retinal explant that is cultured in a cell culture media containing B27.

In certain embodiments, the organotypic culture system comprises a porcine or non-human primate retinal explant that is in a medium that contains Neurobasal A, B27, GlutaMAX, penicillin, streptomycin, and amphotericin B. In particular embodiments, the organotypic culture system comprises a medium of complete Neurobasal A supplemented with GlutaMAX, B27, and 100 units/mL of penicillin, 100 μg/mL of streptomycin, and 0.25 μg/mL Amphotericin B.

In certain embodiments, the organotypic culture system comprises a porcine or non-human primate retinal explant that is cultured in a medium that contains one or more growth factors. In particular embodiments, the one or more growth factors are selected from EGF, FGF, BDNF, NGF, adrenomedullin, angiopoietin, autocrine motility factor, BMP, CNTF, LIF, m-CSF, G-CSF, GM-CSF, EFG, ephrin A1, ephrin A2, ephrin A3, ephrin A4, ephrin A5, ephrin B1, ephrin B2, ephrin B3, EPO, FBS, GDNF, neurturin, persphin, artemin, GDF9, HGF, HDGF, insulin, IGF-1, IGF-2, IL-1, IL-2, IL-3, IL-4, IL-5, IL-6, IL-7, KGF, MSF, GDF-8, neuregulin 1, neuregulin 2, neuregulin 3, neuregulin 4, NT-3, NT-4, PGF, PDFG, renalase, TCGF, TPO, TGF-alpha, TGF-beta, TNF-alpha, Wnt-1, Wnt-2, Wnt-2b, Wnt-3, Wnt-3a, Wnt-4, Wnt-5a, Wnt-5b, Wnt-6, Wnt-7a, Wnt-7b, Wnt-8a, Wnt-8b, Wnt-9a, Wnt-10a, Wnt-10b, Wnt-11, and Wnt-16.

Some embodiments contemplate that any media suitable for culturing primary retinal cells, primary neurons, or neuron-like cells can be used with the organotypic culture system of the present invention. These embodiments contemplate examples of such media are well characterized, and that one of skill in the art can readily identify such media suitable for the organotypic culture system of the present invention.

### Expression of Exogenous Polynucleotides

Certain embodiments contemplate that organotypic culture systems of the present invention are useful for the evaluation or characterization of agents, such as exogenous polynucleotides or vectors containing exogenous polynucleotides. These agents may be candidate agents for gene therapy, or these agents may model particular aspects of a candidate for gene therapy. For example, the organotypic culture system may be used to determine if a viral vector containing a specific promoter induces expression of a transgene in retina, and if so, to determine the level or amount of expression of the transgene, and/or to identify the retinal cell-types where the transgene is expressed.

Gene therapy is the process of introducing foreign genomic materials into host cells to elicit a therapeutic benefit. There are two major categories of gene therapy, germline gene therapy and somatic gene therapy. To date, human gene therapy has been limited to somatic cells. Although numerous viral and non-viral gene delivery systems have been developed, no delivery system has been designed that can be applied in gene therapy of all kinds of cell types in vitro and in vivo with no limitation and side effects. Gene therapy may be performed in somatic cells by viral vectors, e.g. retrovirus, adenovirus, adeno associated virus, helper-dependent adenoviral systems, hybrid adenovirus systems, herpes simplex virus, pox virus, lentivirus, or Epstein-Barr virus, and by non-viral systems such as naked DNA, DNA bombardant, modified mRNA, electroporation, hydrodynamic, ultrasound, magnetofection and through chemical means such as cationic lipids, different cationic polymers, lipid polymers.

As used herein, the term “exogenous polynucleotide,” unless otherwise indicated, refers to any polynucleotide or oligonucleotide that that can be delivered, administered, or contacted to a subject, a cultured tissue, or a cultured cell. In some embodiments “Exogenous polynucleotides” include any vector, e.g. a virus that contains the polynucleotide. Thus, in some embodiments, the exogenous polynucleotide is a DNA oligonucleotide, an RNA oligonucleotide, a modified mRNA, an expression cassette, a primary construct, and/or a virus.

In some embodiments, a method of the present invention comprises contacting a porcine or non-human primate retinal explant with an exogenous polynucleotide to determine the expression of the exogenous polynucleotide in a retina. In certain embodiments, the exogenous polynucleotide is administered as a vector, e.g., an expression vector comprising a promoter sequence operatively lined to an expressible polynucleotide sequence, e.g. a polynucleotide sequence, encoding a therapeutic polypeptide and/or a detectable polypeptide. In particular embodiments, the polynucleotide is contained within a virus, i.e. a viral vector. In some embodiments, the virus is a retrovirus, adenovirus, adeno associated virus, helper-dependent adenoviral systems, hybrid adenovirus systems, herpes simplex virus, pox virus, lentivirus, or Epstein-Barr virus. In some embodiments the polynucleotide is contained as a non-viral vector. In particular embodiments, the non-viral vector is a polynucleotide, including, but not limited to, an expression cassette, a primary construct, DNA, cDNA, mRNA, modified mRNA, siRNA, shRNA, miRNA, oligonucleotide, or morpholino RNA.

Adeno-associated viruses (AAV) are generally small non-enveloped single-stranded DNA viruses. They are non-pathogenic human parvoviruses and are dependent on helper viruses, including adenovirus, herpes simplex virus, vaccinia virus and CMV, for replication. Exposure to wild-type (wt) AAV is not associated or known to cause any human pathologies and is common in the general population, usually occurring in the first decade of life in association with an adenoviral infection [Blacklow et al. 1968; Moskalenko et al. 2000; hereby incorporated by reference in its entirety]. AAV encodes rep and cap genes. The rep gene is required for viral replication and the cap gene is required for synthesis of capsid proteins. Through a combination of alternative translation start and splicing sites, the small genome is able to express four rep and three cap gene products. The rep gene products and sequences in the inverted terminal repeats (145 bp ITRs, which flank the genome) are critical in this process. To date, 1 1 serotypes of AAV have been isolated. AAV2 is the best characterized serotype and the serotype for which most gene transfer studies have been based upon. In particular embodiments, a porcine or non-human primate retinal explant is contacted with an AAV virus. In certain embodiments the serotype of the AAV virus is AAV2.

Replication-defective recombinant adenoviral vectors can be produced in accordance with known techniques. See, Quantin, et al., Proc. Natl. Acad. Sci. USA, 89:2581-2584 (1992); Stratford-Perricadet, et al, J. Clin. Invest, 90:626-630 (1992); and Rosenfeld, et al, Cell, 68: 143-155 (1992); which are hereby incorporated by reference.

rAAVs are small non-enveloped single-stranded DNA viruses. They are nonpathogenic human parvoviruses and are dependent on helper viruses, including adenovirus, herpes simplex virus, vaccinia virus and CMV, for replication.

Examples of viral vectors are those derived from adenovirus (Ad), adeno-associated virus (AAV), lentivirus, or retrovirus. Both human and non-human viral vectors can be used and the recombinant viral vector can be replication-defective in humans. Where the vector is an adenovirus, the vector can comprise a polynucleotide having a promoter operably linked to a gene encoding a polypeptide, e.g. a therapeutic and/or reporter polypeptide, and is replication-defective in humans. Particular embodiments contemplate that virus of any AAV serotype is suitable for use in the organotypic culture system of the present invention. In certain embodiments, AAV vectors with modified caspids are suitable for use in the organotypic culture system of the present invention.

### Methods of Generating Organotypic Retina Culture

Certain embodiments of the present invention relate to methods of culturing a porcine or non-human primate retinal explant in an organotypic culture system. Generally, such methods are performed by isolating a portion of retina i.e. a retinal explant containing live cells, from the eye of a pig or a non-human primate, contacting the porcine or non-human primate retinal explant with a cell culture medium; and incubating the porcine or non-human primate retinal explant in an organotypic culture system for a period of time. Some embodiments contemplate that the organotypic culture system of the present invention is useful for, inter alia, determining the expression of an exogenous polynucleotide in retinal tissue. In some embodiments, expression of an exogenous polynucleotide in retinal tissue may be assessed by contacting the retinal explant prior to or during the process of culturing the porcine or non-human primate retinal explant in the organotypic culture system. Alternatively, the porcine or non-human primate retinal explant may be contacted with the exogenous polynucleotide after the porcine or non-human primate retinal explant has been established in the organotypic culture system of the present invention.

In particular embodiments, a porcine or non-human primate retinal explant is contacted with an exogenous polynucleotide to determine the expression of the exogenous polynucleotide in retina. Without being bound by theory, certain embodiments contemplate that the use of the organotypic pig or non-human primate retina culture systems of the present invention to determine the expression of an exogenous polynucleotide in retina has several advantages over other model systems. Pig and non-human primate retina is similar to the human retina, in that all ten of the distinct, anatomical layers of the human retina are present in the pig and non-human primate retinas, which is not the case for other animal retinas, such as dog, cat, and rodent retinas. Another advantage is that exogenous polynucleotides can be evaluated quickly in the present system with a higher degree of precision and control that can be obtained with in vivo animal studies. The organotypic pig retina culture system has advantages over other retina or retinal cell culture systems, in that the present system provides an intact tissue and cellular morphology that persists over a period of at least 2 to 4 weeks, longer than the typical retinal culture systems, which is suitable to evaluate exogenous polynucleotide expression, including viral vectors. The many types of cells present in the retina are found within the porcine or non-human primate retinal explant of the present invention, and this diversity of cells remain viable for a period of weeks.

Advantages of the organtotypic culture system of the present invention include the speed in which experiments may be performed. For example, expression of an exogenous polynucleotide, e.g. a virus, may be assessed within two weeks after exposing the retinal tissue to the virus. Another advantage of the present invention is that is allows for experiments to be performed in tightly controlled conditions, reducing the number of animals required to achieve statistically meaningful results.

Particular embodiments contemplate that organotypic cell culture system of the present invention has advantages over retinal cell culture systems. For example, dissociated cell culture systems are difficult to maintain as only one or two cell types can be grown. Many cell culture systems utilize retinal cells derived from rodents, and both pigs and non-human primates are more closely related to humans, have more closely related genomes (and therefore similar transcriptional elements), have similarly large number of photoreceptors to humans than rodents. Alternative systems such as induced pluripotent stem cell and embryonic stem cell derived eye cups take very long to mature and are more expensive to generate than the organotypic culture system of the present invention.

One of skill in the art will understand that methods of generating organotypic cultures from primary tissues should be performed under sterile conditions where possible. For example, one of skill in the art will understand that when obtaining a tissue from an animal, it is often not possible for the animal itself to be sterile, but that any tools, e.g. surgical tools and pipet tips, reagents, e.g. dissection medium and culture medium, surfaces or hoods where tissue dissection takes place, and hoods and incubators where organotypic cultures are housed or placed, can and should be sterilized. Thus, in some embodiments, methods of the present invention are performed in sterile or near sterile conditions.

Particular embodiments of the present invention relate to in vitro methods of determining the expression of an exogenous polynucleotide, such as a gene therapy vector, in the retina. Certain embodiments are directed to methods of determining expression of a exogenous polynucleotide in a porcine or non-human primate retinal explant, wherein the method is performed by isolating a portion of retina from a pig eye containing live cells; contacting the portion of the retina to the exogenous polynucleotide; incubating the portion of the retina in an organotypic culture system for a period of time; and determining the expression of the exogenous polynucleotide in the retinal explant; thereby determining the expression of the polynucleotide.

Certain embodiments contemplate that the organtogypic culture system and the methods of the present invention are useful for the evaluation of specific features of an exogenous polypeptide. For example, the present organotypic culture systems and methods may be used to evaluate features of a virus, such as expression or cellular tropism resulting from different promoters, viral serotypes, enhancer elements, and any cofactor that may be administered with this virus.

### Isolating a Retinal Explant

Particular embodiments contemplate that isolating a porcine retinal explant for culturing in the organotypic culture system of the present invention includes the steps of obtaining a pig eye from a pig, removing the retina from the pig eye, and isolating a portion of the retina for culturing in the organotypic culture system of the present invention.

In some embodiments, the pig eye is removed from the pig within about 24 hours, within about 8 hours, within about 6 hours, within about 5 hours, within about 4 hours, within about 3 hours, within about 2 hours, or within about 1 hour postmortem. In particular embodiments, the pig eye is enucleated from the pig within about 180 minutes, about 150 minutes, about 120 minutes, about 90 minutes, about 60 minutes, about 45 minutes, about 30 minutes, or less than 30 minutes postmortem. In certain embodiments, the pig eye is removed from the pig within 2 hours post mortem.

In certain embodiments, the pig eye is removed from a miniature pig. In some embodiments, the miniature pig is a Yucatan pig, a Yucatan micro pig, a Sinclair pig, or a Göttingen pig. In particular embodiments, the pig eye is obtained from a domestic pig. In particular embodiments, the pig is an American Landrace pig, an American Yorkshire pig, a Berkshire pig, a Mulefoot pig, a Tamworth pig, a Large Black pig, a Hereford pig, a Poland China pig, a Chester white pig, a Landrace pig, a Hampshire pig, a Duroc pig, or a Yorkshire pig. In some embodiments, the eye is removed from an American Yorkshire pig.

Particular embodiments contemplate that isolating a non-human primate retinal explant for culturing in the organotypic culture system of the present invention includes the steps of obtaining an eye from a non-human primate, removing the retina from the eye, and isolating a portion of the retina for culturing in the organotypic culture system of the present invention.

In some embodiments, the non-human primate eye is removed from the non-human primate within about 24 hours, within about 8 hours, within about 6 hours, within about 5 hours, within about 4 hours, within about 3 hours, within about 2 hours, or within about 1 hour postmortem. In particular embodiments, the non-human primate eye is enucleated from the non-human primate within about 180 minutes, about 150 minutes, about 120 minutes, about 90 minutes, about 60 minutes, about 45 minutes, about 30 minutes, or less than 30 minutes postmortem. In certain embodiments, the non-human primate eye is removed from the non-human primate within 2 hours postmortem.

In certain embodiments, the eye is removed from a non-human primate. In particular embodiments, the eye is obtained from a non-human primate that is a macaque (e.g. a Rhesus macaque or a cynomolgous macaque), a marmoset, a tamarin, a spider monkey, an owl monkey, a vervet monkey, a squirrel monkey, a baboon, or an orangatang. In particular embodiments, the organotypic culture system contains non-human primate retinal explant that is obtained from an eye of an adult non-human primate.

In some embodiments, the pig or non-human primate eye is placed in cold solution once it is removed from the pig or the non-human primate. The pig eye remains in solution until the retina can be removed from the eye. Thus, if the eye is removed in one facility but the retina will be removed in a second facility, the eye will remain in solution until the eye can be transported to the second facility. Some embodiments contemplate that one of skill in the art will readily identify solutions that are appropriate for storage or transport of an eye, and include buffers such as phosphate buffered saline (PBS), CO₂-independent medium, cell culture basal media such as Neurobasal, Neurobasal A, DMEM, or MEM. In some instances, the solution will have added agents to improve cell survival and tissue integrity, including, but not limited to, chemicals known to prevent apoptosis in cells, including neurons, and/or protease inhibitors. In some embodiments, the solution is cold. In certain embodiments, the solution is at a temperature of between 0° C. and 10° C., between 2° C. and 10° C., between 4° C. and 10° C., or about 1° C., about 2° C., about 3° C., about 4° C., about 5° C., about 6° C., about 7° C., about 8° C., about 9° C., or about 10° C. In particular embodiments, the eye is removed from the pig and placed in cold PBS at a temperature between 4° C. and 10° C.

In particular embodiments, the retina is removed from the eye within about 24 hours, within about 8 hours, within about 6 hours, within about 5 hours, within about 4 hours, within about 3 hours, within about 2 hours, or within about 1 hour after the eye is enucleated from the pig. In particular embodiments, the retina is removed within about 180 minutes, about 150 minutes, about 120 minutes, about 90 minutes, about 60 minutes, about 45 minutes, about 30 minutes, or less than 30 minutes after the eye is enucleated from the pig. In certain embodiments, the retina is removed from the pig eye within 2 hours after the eye is enucleated from the pig. In some embodiments, the retina is removed from the non-human primate eye within 2 hours after the eye is enucleated from the non-human primate.

Some embodiments contemplate that the retina can be removed from the eye ball by any means known in the art, so long as the retina can be removed from the eye while remaining in a condition that is viable for use in the organotypic culture.

In some embodiments, extraneous tissue is removed from the eye prior to removing the retina from the eye. The extraneous tissue may be removed with a knife, such as a surgical scalpel, or with surgical scissors, or any tool known by one of skill in the art to be suitable for removing extraneous tissue from the eye without damaging the eye.

In certain embodiments, the retina is removed from the eye through the steps of cutting the eye along the corneal limbus of the eye into an anterior portion and a posterior portion, wherein the posterior portion contains the optic disc and the retina; placing the posterior portion into a dissection medium; and removing the retina and placing it into dissection medium; thereby isolating the retina from the pig or non-human primate eye.

In particular embodiments, the retina is removed from the eye by cutting the eyeball along the corneal limbus into anterior and posterior portions. The posterior portion (eyecup) is transferred into a petri dish containing dissection medium, and the vitreous humor is carefully removed from the eyecup, leaving the retinal attached to the eyecup. The optic disk is severed and the retina is then peeled off and placed into a petri dish containing dissection media.

In particular embodiments, the dissection medium is a cell culture medium, PBS, or CO₂-independent medium. In certain embodiments, the dissection medium is CO₂-independent medium. In some embodiments, the dissection medium is cold. In certain embodiments, the dissection medium is at a temperature of between 0° C. and 10° C., between 2° C. and 10° C., between 4° C. and 10° C., or about 1° C., about 2° C., about 3° C., about 4° C., about 5° C., about 6° C., about 7° C., about 8° C., about 9° C., or about 10° C. In particular embodiments, the eye is removed from the pig or the non-human primate and placed in cold PBS at a temperature between 4° C. and 10° C.

CO₂-independent medium used for supporting cell growth for a variety of suspension and adherent mammalian cells such as epithelial, fibroblast, and lymphoid cell lines without a CO₂ incubator. CO₂—Independent Medium is suitable for transporting cells or tissue and for the handling of mouse embryos under atmospheric conditions. One of skill in the art will recognize suitable CO₂-independent medium for the methods of the present invention, which optionally include media described by Vistica et al. J Natl Cancer Inst. 1990 Jun. 20; 82(12):1055-61; herein incorporated by reference in its entirety.

In some embodiments, the CO₂-independent medium is a CO₂-independent medium manufactured by Gibco. CO₂-independent medium contains sodium pyruvate and phenol red, and lacks L-glutamine and HEPES. CO₂-independent medium contains a unique buffering system composed of mono and dibasic sodium phosphate and β-glycerophosphate. A small amount of sodium bicarbonate is included in the formulation to meet essential bicarbonate dependent functions. No synthetic buffers are present, which eliminates any cytotoxic effects associated with such buffering systems. Additionally, CO₂-independent medium has been formulated with components that enhance cellular production and utilization of CO₂ such that an exogenous source of CO₂ is not required for the maintenance of CO₂-dependent cellular functions is manufactured by GIBCO and is a medium

Particular embodiments contemplate that a porcine or non-human primate retinal explant for use in the organotypic culture system can be removed from a retina using any method known in the art, including dissection with surgical tools, for example a knife or scalpel, or with a tissue punch. In certain embodiments, a retinal explant that is circular in shape is removed from the pig or non-human primate retina. In some embodiments, the retinal explant is cut from the retina and has a diameter of about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, or about 16 mm. In particular embodiments, the non-human primate retinal explant has a diameter of between about 100 μm and about 20 mm, between about 500 μm and about 16 mm, between about 1 mm and about 16 mm, between about 2 mm and about 8 mm, or between about 4 mm and about 6 mm. In certain embodiments, the non-human primate retinal explant is about 4 mm to about 16 mm in diameter.

In particular embodiments, the porcine or non-human primate retinal explant is removed from the retina in a manner that leaves at least a portion of the defined morphological layers of the retina. In some embodiments, the retinal explant is isolated from the retina in a manner that leaves more than one of, at least two of, at least three of, at least four of, at least five of, at least six of, at least seven of, at least eight of, at least nine of, or all ten of the distinct, morphological layers of the retina intact. In particular embodiments, the procine retinal explant is removed from the retina and the retinal explant maintains all of the nerve fiber layer, the ganglion cell layer, the inner plexiform layer, the inner nuclear layer, the outer plexiform layer, the outer nuclear layer, the external limiting membrane, and the layer of rods and cones. In certain embodiments, the non-human primate retinal explant is removed from the retina and the retinal explant maintains all of the nerve fiber layer, the ganglion cell layer, the inner plexiform layer, the inner nuclear layer, the outer plexiform layer, the outer nuclear layer, the external limiting membrane, the layer of rods and cones, and the retinal pigment epithelium.

In some embodiments, the porcine or non-human primate retinal explant is placed in a container suitable for organotypic culture after it is removed from the pig retina. In some embodiments, the container is a cell culture dish, a well of a cell culture plate, or a transwell that is inserted into a cell culture dish or a well of a cell culture plate.

In some embodiments, the porcine or non-human primate retinal explant is placed in a cell culture dish that has a circular shape after it is removed from the pig retina. In particular embodiments, the porcine or non-human primate retinal explant is placed in a circular cell culture dish that has a diameter of 35 mm, 60 mm, 100 mm, 120 mm, 150 mm, or 200 mm, after it is removed from the pig retina.

In some embodiments, the porcine or non-human primate retinal explant is placed in a well of a cell culture plate after it is removed from the pig retina. In particular embodiments, porcine or non-human primate retinal explant is placed in the well of a single well plate, a 2 well plate, a 6 well plate, a 12 well plate, a 24 well plate, a 48 well plate, or a 96 well plate after it is removed from the pig retina.

In particular embodiments, the porcine or non-human primate retinal explant is placed in a transwell after it is removed from the pig retina. The transwells have a top compartment made up of a circular wall and a porous bottom membrane, so that when the transwell is placed in a well with a medium, tissues placed in the top compartments are in contact with medium through the pores in the membrane, provided the well is filled with enough medium to contact the membrane. Further, the transwell allows for a cultured tissue or cultured cells to be exposed to different conditions simultaneously by contacting a bottom surface of the tissue with one medium and filling the top compartment with a different medium, or even no medium. A further advantage is that the transwell system can allow an experimenter to control which surface or portion of a tissue will contact a media.

In some embodiments, the porcine or non-human primate retinal explant the is placed into a transwell after it is removed from the pig retina, wherein the transwell is sized for placement in a well of a 6 well plate, a 12 well plate, a 24 well plate, a 48 well plate, or a 96 well plate. In particular embodiments, the porcine or non-human primate retinal explant is placed into a transwell that is 1 mm, 2 mm, 3 mm, 4 mm, 5 mm, 6 mm, 7 mm, 8 mm, 9 mm, 10 mm, 11 mm, 12 mm, 15 mm, 18, mm, 20 mm, 24 mm, 30 mm, 35 mm, 40 mm, 45 mm, 50 mm, 60 mm, 70 mm, 80 mm, 90 mm, or 100 mm in diameter. In particular embodiments, the porcine or non-human primate retinal explant is placed into the top compartment of a transwell that contains a porous membrane with 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.4 μm, 1.6 μm, 1.8 μm, 2 μm, 3 μm, or 4 μm pore size.

In certain embodiments, the porcine or non-human primate retinal explant is placed into a transwell after it is removed from the pig retina, so that the surface of the porcine retinal exposing the photoreceptors is placed in direct contact with the porous bottom membrane of the transwell, and the opposite surface of the retinal explant is exposed is facing upwards. In some embodiments, the porcine retinal explant is placed into the transwell by floating the retinal explant onto an amount of dissection medium in the top compartment of the transwell with the photoreceptor layer facing downin a transwell with a 0.4 μm pore polycarbonate membrane. Once the retinal explants settle flat onto the transwell membrane, the dissection media is removed.

Certain embodiments contemplate that the organotypic pig or non-human primate retinal culture system is suitable to test the expression of exogenous polynucleotides in retina, including polynucleotides that are candidate gene therapy agents. In particular embodiments, the isolated porcine retinal explant is contacted with the polynucleotide before or during the process of culturing the retinal explant in the organotypic culture system of the present invention. In some embodiments, the porcine retinal explant is contacted with a polynucleotide after the porcine retinal explant has been established in the organotypic culture system.

In particular embodiments, the exogenous polynucleotide is placed into a solution, and the porcine or non-human primate retinal explant is contacted with the solution containing the exogenous polynucleotide to transfect the porcine or non-human primate retinal explant with the polynucleotide. In particular embodiments, the solution is a cell culture medium. In certain embodiments, solution is a culture medium suitable for retinal cell culture. In particular embodiments, the solution contains the same culture medium as the organotypic retinal culture medium.

In certain embodiments, the exogenous polynucleotide in the solution is a virus. In some embodiments, the virus in solution is a retrovirus, an adenovirus, an AAV, a helper-dependent adenoviral systems, a hybrid adenovirus systems, a herpes simplex virus, a pox virus, a lentivirus, or an Epstein-Barr virus. In some embodiments the polynucleotide in solution is a non-viral polynucleotide that is not associated with a virus. In particular embodiments, the non-viral polynucleotide is an expression cassette, a primary construct, DNA, a cDNA, an mRNA, a modified mRNA, an siRNA, an shRNA, an miRNA, an oligonucleotide, or a morpholino RNA.

In particular embodiments, the porcine or non-human primate retinal explant is contacted with a solution containing the exogenous polynucleotide, wherein the exogenous polynucleotide is a virus, with an amount of the virus of about 1×10⁻³ to about 1×10⁹ multiplicity of infection (MOI). Methods of calculating the multiplicity of infection are known in the art and include, but are not limited to, calculating the cell number of the pig retinal explant by dissociating a pig retinal cell explant into single cell suspensions using, such as with the Papain Dissociation System, and performing cell counts, such as with a hemocytometer, to estimate the total yield of cells from a single explant. In certain embodiments, the retinal pig explant is contacted with a solution having an amount of the virus of about 1×10⁻² to about 1×10¹⁰ MOI, about 1×10⁻¹ to about 1×10¹¹ MOI, about 1×10⁰ to about 1×10¹² MOI, about 1×10¹ to about 1×10¹⁰ MOI, about 1×10² to about 1×10⁸ MOI, about 1×10³ to about 1×10⁹ MOI, about 1×10³ to about 1×10⁸ MOI, about 1×10³ to about 1×10⁸ MOI, about 1×10³ to about 1×10⁷ MOI, or about 1×10⁴ to about 1×10⁶ MOI. In some embodiments, the porcine or non-human primate retinal explant is contacted with a solution having an amount of the virus of about 1×10¹ MOI, about 1×10² MOI, about 1×10³ MOI, about 5×10³ MOI, about 1×10⁴ MOI, about 2×10⁴ MOI, about 3×10⁴ MOI, about 4×10⁴ MOI, about 5×10⁴ MOI, about 6×10⁴ MOI, about 7×10⁴ MOI, about 8×10⁴ MOI, about 9×10⁴ MOI, about 1×10⁵ MOI, about 5×10⁵ MOI, or about 1×10⁶ MOI.

In certain embodiments, the porcine or non-human primate retinal explant is contacted with a solution containing the polynucleotide that is a virus with a concentration of the virus of about 1×10¹ to about 1×10¹⁴, about 1×10² to about 1×10¹³, about 1×10³ to about 1×10¹², about 1×10⁴ to about 1×10¹⁰, or about 1×10⁶ to about 1×10⁸ viral genomes per microliter. In some embodiments, the porcine or non-human primate retinal explant is contacted with a solution containing a concentration of about 1×10⁴, about 5×10⁴, about 1×10⁵, about 5×10⁵, about 1×10⁶, about 5×10⁶, about 1×10⁷, about 5×10⁷, about 1×10⁸, about 2×10⁸, about 3×10⁸, about 4×10⁸, about 5×10⁸, about 6×10⁸, about 7×10⁸, about 8×10⁸, about 9×10⁸, 1×10⁹, about 5×10⁹, or about 1×10¹⁰ viral genomes per microliter.

In certain embodiments, the porcine or non-human primate retinal explant is contacted with about 100 microliters of solution containing an amount of exogenous polynucleotide that is a virus of about 1×10³ to about 1×10¹⁶, about 1×10⁴ to about 1×10¹⁵, about 1×10⁴ to about 1×10¹⁴, about 1×10⁶ to about 1×10¹², or about 1×10⁸ to about 1×10¹⁰ polynucleotide genomes per microliter. In some embodiments, the porcine or non-human primate retinal explant is contacted with a solution containing the a concentration of about 1×10⁴, about 5×10⁴, about 1×10⁵, about 5×10⁵, about 1×10⁶, about 5×10⁶, about 1×10⁷, about 5×10⁷, about 1×10¹⁰, about 2×10¹⁰, about 3×10¹⁰, about 4×10¹⁰, about 5×10¹⁰, about 6×10¹⁰, about 7×10¹⁰, about 8×10¹⁰, about 9×10¹⁰, 1×10¹¹, about 5×10¹¹, or about 1×10¹² viral genomes per 50, 100, 150, 200, 250, 300, 400, or 500 microliters.

In some embodiments, the porcine or non-human primate retinal explant is placed in a cell culture dish or well and is contacted with a solution containing the polynucleotide. In particular embodiments, the pig retinal explant is placed in a transwell and is contacted with a solution containing the polynucleotide. In certain embodiments, the pig retinal explant is placed in the top compartment of a transwell with the photoreceptor side of the pig retinal explant contacting the membrane of the transwell and the solution containing the polynucleotide is added to the top compartment of the transwell. In some embodiments, the porcine or non-human primate retinal explant is contacted with the exogenous polynucleotide after the retina explant has been removed from the retinal but before the retinal explant has been cultured, established in the organotypic culture system, and/or contacted with cell culture medium. In particular embodiments, the retinal explant is contacted with the exogenous polypeptide after it has been cultured, established in the organotypic culture system, and/or contacted with cell culture medium.

In some embodiments, the porcine or non-human primate retinal explant is cultured in a cell culture dish or well and is contacted with a solution containing the exogenous polynucleotide. In certain embodiments, the pig retinal explant is cultured in a cell culture dish or well, and the culture medium is replaced with a solution containing the exogenous polynucleotide. In certain embodiments, the pig retinal explant is cultured in a cell culture dish or well, and the solution containing the exogenous polynucleotide is added to the culture medium. In particular embodiments, the pig retinal explant is cultured in a transwell and is contacted with a solution containing the exogenous polynucleotide. In certain embodiments, the pig retinal explant is cultured in the top compartment of a transwell with the photoreceptor side of the pig retinal explant contacting the membrane of the transwell and the solution containing the exogenous polynucleotide is added to the top compartment of the transwell.

In particular embodiments, the porcine or non-human primate retinal explant is contacted with the exogenous polynucleotide or a solution containing the exogenous polynucleotide for about 5 minutes, about 10 minutes, about 15 minutes, about 20 minutes, about 25 minutes, about 30 minutes, about 45 minutes, about 60 minutes, about 2 hours, about 3 hours, about 4 hours, about 5 hours, about 6 hours, about 8 hours, about 12 hours, about 18 hours, or about 24 hours.

### Incubation

In certain embodiments, culturing the pig retinal explant involves the steps of placing the porcine or non-human primate retinal explant in a cell culture dish, in a well of a cell culture plate, or in a transwell and adding an organotypic culture medium to the dish, well, or transwell. In particular embodiments, the cell culture medium comprises a basal medium. In some embodiments, the basal medium is Neurobasal, Neurobasal A, DMEM, RPMI 1640, MEM, and/or IMDM basal medium. In some embodiments, the medium contains a neuron cell culture supplement. In certain embodiments, the medium contains an L-glutamine supplement, a neuron supplement, and at least one antibiotic. In particular embodiments, the neuron cell culture supplement is B27, N2, or BIT9500. In certain embodiments, the medium contains Neurobasal A, B27, GlutaMAX, penicillin, streptomycin, and amphotericin B. In particular embodiments, the organotypic culture system comprises a medium of complete Neurobasal A supplemented with GlutaMAX, B27, and 100 units/mL of penicillin, 100 μg/mL of streptomycin, 0.25 μg/mL Amphotericin B, and growth factor, e.g. FGF and/or EGF.

In particular embodiments, porcine or non-human primate retinal explants are cultured in a medium containing complete Neurobasal A media supplemented with GlutaMAX, B27 supplement, and 100 units/mL of penicillin, 100 μg/mL of streptomycin, and 0.25 μg/mL Amphotericin B, which is added into the well under the transwell insert and filled up to the level of the transwell membrane so that the medium contacts the bottom surface of the tissue through the pores of the membrane and the top surface of the tissue is exposed to the atmosphere.

In certain embodiments, porcine or non-human primate retinal explants are maintained at 37° C. in a 5% CO2 humidified environment for up to 3 days, 4 days, 5 days, 6 days, 1 week, 2 weeks, 3 weeks, 4 weeks, 5 weeks, or six weeks. In particular embodiments, a media change is performed every 48 hours.

Particular embodiments contemplate that the porcine or non-human primate retinal explant can be cultured for a period of time, during in which at least a portion of the cells in the porcine or non-human primate retinal explant remain viable and/or retains cellular physiology. In some embodiments, the period of time where the portion of cells remain viable and/or retain cellular physiology is at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, or at least 42 days.

Certain embodiments contemplate that the porcine or non-human primate retinal explant can be cultured for a period of time, during which the distinct, anatomical of the porcine or non-human primate retinal explant retain at least a portion of their live cells and/or retain their distinct physiology. In some embodiments, the period of time in which the layers retain lives cells and/or retain their physiology is at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, or at least 42 days.

### Detecting Polypeptides

In certain embodiments, the retinal pig explant is contacted with an exogenous polynucleotide, and the retinal pig explant is examined to determine the expression of the exogenous polynucleotide in a pig retina. In some embodiments, the retinal pig explant is examined to predict the expression of the polynucleotide in a retina, such as a human retina. In some embodiments, the exogenous polynucleotide is a candidate gene therapy vector. In some embodiments, the retinal pig explant is contacted with an exogenous polynucleotide that is a virus, such as a retrovirus, an adenovirus, an adeno-associated virus (AAV), a helper-dependent adenoviral virus, a hybrid adenovirus virus, a herpes simplex virus, a pox virus, a lentivirus, or an Epstein-Barr virus, and the retinal pig explant is examined to demine the expression of the exogenous polynucleotide in the pig retina and/or predict expression of the polynucleotide in a retina, such as a human retina. In certain embodiments, the retinal pig explant is contacted with an exogenous polynucleotide that is not a virus, including, but not limited to, an expression cassette, a primary construct, DNA, cDNA, mRNA, modified mRNA, siRNA, shRNA, miRNA, oligonucleotide, or morpholino RNA, and the retinal explant is examined to demine the expression of the polynucleotide in the porcine or non-human primate retinal explant and/or predict expression of the polynucleotide in a retina, such as a human retina.

In particular embodiments, the porcine or non-human primate retinal explant is contacted with an AAV comprising a polynucleotide, and the retinal pig explant is examined to determine the polynucleotide expression in the retina. In certain embodiments, the AAV is AAV1, AAV2, AAV2.5, AAV3, AAV4, AAV5, AAV6, AAV7, AAV8, AAV9, AAV10, AAV11, AAV 12, and hybrids or variants, including mutated or otherwise non-naturally occurring variants, thereof.

In some embodiments, the retinal pig explant is contacted with a polynucleotide, and the retinal pig explant is examined to determine the expression of the polynucleotide after a sufficient amount of time for the polynucleotide do be expressed by a cell in the retinal pig explant. In some embodiments, the amount of time sufficient for a polynucleotide to be expressed by a cell in the retinal pig explant is 6 hours, 12 hours, 18 hours, 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 35 days, or 42 days after the retinal pig explant is contacted with the polynucleotide. In some embodiments, the amount of time sufficient for a polynucleotide to be expressed in the pig retinal explant is between 1 hour and 6 hours, between 6 hours and 48 hours, between 12 hours and 48 hours, between 24 hours and 48 hours, between 3 days and 42 days, between 3 days and 28 days, between 3 days and 14 days, between 3 days and 10 days, between 3 days and 7 days, between 5 days and 10 days, between 7 days and 14 days, between 7 days and 21 days, between 14 days and 21 days, between 21 and 28 days, and/or between 21 and 48 days after the retinal pig explant is contacted with the polynucleotide.

In particular embodiments, the retinal pig explant is contacted with a polynucleotide that expresses a polypeptide, and the retinal pig explant is examined to determine the expression of the polynucleotide by detecting the presence and/or an amount of the polypeptide. In certain embodiments, the polynucleotide encodes a therapeutic polypeptide, a reporter polypeptide, a fusion polypeptide, and/or a secreted polypeptide.

In some embodiments, the retinal pig explant is contacted with an exogenous polynucleotide that encodes a polypeptide that is a reporter polypeptide or a fusion polypeptide that comprises a reporter polypeptide. In some embodiments, the reporter polypeptide is a fluorescent reporter polypeptide. Fluorescent reporter polypeptides are well known in the art, and include, but are not limited to TagBFP, mTagBFP2, Azurite, EBFP2, mKalama1, Sirius, Sapphire, T-Sapphire, ECFP, Cerulean, SCFP3A, mTurquoise, mTurquoise2, monomeric Midoriishi-cyan, TagCFP, mTFP1, GFP, EGFP, Emeral, Superfolder GFP, monomeric Azami Green, TagGFP2, mUKG, mWasabi, Clover, mNeonGreen, EYFP, YFP, Citrine, Venus, SYFP2, TagYFP, monomeric Kusabira Orange, MKOK, mKO2, mOrange, mOrange2, mRaspberry, mCherry, mStrawberry, mTangerine, tdTomato, TagRFP, TagRFP1, mApple, mRuby, mRuby2, TagRFP675, IFP1.4, iFRP, mKeima Red, LSS-mKate1, LSS-mKate2, mBeRFP, PA-GFP, PAmCherry1, PATagRFP, Kaede green, Kaede red, KikGR1 green, KikGR1 red, PS-CFP2, mEos2 green, mEos2 red, mEos3.2 green, mEos3.2 red, and PSmOrange.

In particular embodiments, the porcine or non-human primate retinal explant is contacted with an exogenous polynucleotide that comprises a sequence that encodes a non-fluorescent reporter polypeptide or a fusion polypeptide that comprises a non-fluorescent reporter polypeptide. In certain embodiments, the reporter polypeptide is a non-fluorescent reporter polypeptide. In some embodiments, the non-fluorescent reporter polypeptide is a peptide tag. Peptide tags include, but are not limited to, AviTag, Calmodulin-tag, a polynucleotide polyglutamate tag, E-tag, FLAG-tag, FC-tag, HA-tag, His-tag, Myc-tag, S-tag, SBP-tag, Softag 1, Softag 3, Strep-tag, Strep-tag II, TC tag, V5 tag, VSV-tag, Xpress tag, Isopeptag, SpyTag, and Snooptag. In particular embodiments, the non-fluorescent reporter polypeptide is a full length polypeptide. Non-fluorescent polypeptides are known in the art, and include, but are not limited to, biotin carboxyl carrier protein, Glutathione-S-transferase, Halo-tag, β-galactosidase, β-galactosidase, chloramphenicol acetyltransferase, recombinant luciferase, and secreted alkaline phosphatase.

In particular embodiments, the retinal pig explant is contacted with a polynucleotide that encodes a therapeutic polypeptide. In some embodiments, the therapeutic polypeptide is an anti-angiogenic factor, including but not limited to sFlt-1, sFLTOl, VEGF trap, an anti-VEGF antibody or antibody fragment, an anti-VEGF soluble receptor or receptor fragment, an Fc fusion protein comprising a soluble FLT peptide or fragment, soluble FLT peptides or fragments, PEDF, angiostatin, endostatin, TIMP3, or PDGF inhibitor. In some embodiments, the therapeutic polypeptide is a neurotrophic or anti-apoptotic factor. Neurotrophic or anti-apoptotic factors include, but are not limited to, GDNF, CNTF, BDNF, NTN, NT-4, NGF, or RdCVF, PDGF-R. In certain embodiments, the therapeutic protein modulates a genetic disease, and includes, but is not limited to, RPE65, retinoschisin, CRB1, retinitis pigmentosa GTPase regulator (RGPR)-interacting protein-1, peripherin, peripherin-2 (Prph2), a light-responsive opsin (ChR2; Chop2, L-opsin (OPN1LW), M-opsin (OPNIMW), S-Opsin (OPNISW), rhodopsin (Rhl, OPN2, RHO), fibroblast growth factor 2, nurturin, epidermal growth factor or complement inhibitor. In particular embodiments, the therapeutic polypeptide is soluble. In particular embodiments, the therapeutic protein is VEGFR-1

In particular embodiments, the retinal pig explant is contacted with an exogenous polynucleotide that encodes a polypeptide, and the presence and/or the amount of the polypeptide present in the porcine or non-human primate retinal explant is determined by assessing the expression of the encoded polypeptide. Methods of detecting protein in a tissue are well known in the art, and include, but are not limited to live cell imaging, microscopy, fluorescence microcopy, immunoelectrophoresis, immunohistochemistry, immunocytochemistry, immunofluorescence, western blotting, and ELISA. In particular embodiments, the retinal pig explant is contacted with a polynucleotide, and the expression of the polynucleotide is assessed by detecting the polypeptide expressed by the polynucleotide by live cell imaging, microscopy, fluorescence microcopy, immunoelectrophoresis, immunohistochemistry, immunocytochemistry, immunofluorescence, western blotting, and/or ELISA.

In some embodiments, the porcine or non-human primate retinal explant is contacted with an exogenous polynucleotide that encodes a polypeptide, and the expression of the polynucleotide is assessed by determining the cellular tropism of the polynucleotide, i.e., identifying retinal cell types that express the polypeptide encoded by the polynucleotide. Determining the cellular tropism of a polynucleotide can be achieved by techniques known in the art, including but not limited to double or multi-label immunofluorescence. Double or multi-label immunofluorescence is an established tool for research purposes that can be utilized where there is a need for knowledge about the relative localization of a target polypeptide that can only be achieved by visualizing several targets in one slide. For example, the cellular tropism of a polynucleotide can be determined by labeling the polypeptide encoded by the polynucleotide with a primary and secondary antibody combination that binds to and labels the polypeptide with a fluorescent tag, and also staining a marker polypeptide associated with a specific cell type with a primary and secondary combination that labels the marker polypeptide with a different fluorescent tag. It is important that the secondary antibodies contain fluorescent tags that have spectral properties that are distinguishable from each other.

Double and multi-label immunofluorescence techniques are well characterized, and one of skill in the art can readily select and identify marker polypeptides to identify particular cell populations in the retina, as well as appropriate secondary antibodies that are compatible for double staining and multiple staining fluorescence techniques. Suitable maker polypeptides include, but are not limited to beta-III-tubulin, which is localized to neurons including photoreceptors, bipolar cells, ganglion cells, horizontal cells, and amacrine cells; Chx10, which is localized to bipolar cells; rhodopsin, which localized to rod cells; and L/M opsin, which is localized to cone cells. Suitable secondary antibodies include, but are not limited to, secondary antibodies conjugated to Alexa Flour 350, Alexa Flour 405, Alexa Flour 488, Alexa Flour 532, Alexa Flour 568, Alexa Flour 680, Alexa Flour 647, Alexa Flour 750, FITC, TRITC, Rhodamine, Texas Red, Pacific Orange, Pacific Green, and Pacific Blue.

In particular embodiments, the porcine or non-human primate retinal explant is contacted with a polynucleotide that encodes a polypeptide, and the presence and/or the amount of an mRNA encoding the polypeptide present in the porcine or non-human primate retinal explant is determined to assess the expression of the polynucleotide. Methods of detecting an mRNA in a tissue are well known in the art, and include, but are not limited to, polymerase chain reaction (PCR), e.g., qPCR and rt PCR, and in situ hybridization. In particular embodiments, the retinal explant is contacted with an exogenous polynucleotide, and the expression of the polynucleotide is assessed by detecting the mRNA transcribed by the polynucleotide by PCR or in situ hybridization.

In some embodiments, the retinal pig explant is contacted with an exogenous polynucleotide that decreases the expression of an endogenous polypeptide expressed in the porcine or non-human primate retinal explant, and the expression of the polynucleotide is determined by measuring a decrease in an amount of the endogenous polypeptide. Methods of detecting a decrease in an amount of an endogenous polypeptide in a tissue are well known in the art, and include, but are not limited to, microscopy, fluorescence microcopy, immunoelectrophoresis, immunohistochemistry, immunocytochemistry, immunofluorescence, western blotting, and ELISA. In particular embodiments, the retinal pig explant is contacted with a polynucleotide that reduces the expression of an endogenous polypeptide, and the expression of the polynucleotide is assessed by detecting the amount of the endogenous polypeptide by microscopy, fluorescence microcopy, immunoelectrophoresis, immunohistochemistry, immunocytochemistry, immunofluorescence, western blotting, and/or ELISA.

### EXAMPLES

### Example 1

### Development of Organotypic Porcine Retinal Culture System

While in vivo gene therapy studies provide valuable information regarding vector specificity, therapeutic efficacy, and safety, they can require a significant amount of time to develop and require large sample sizes to account for variability. An ex vivo culture system can provide advantages over in vivo systems for gene therapy studies, allowing for faster experimental turn-around time and requiring fewer replicates to account for variability. As such, an organotypic porcine retinal culture system was developed for use in high-throughput ex vivo analyses and pharmacological screenings. Porcine retinas were selected as their anatomic and physiologic features are similar to humans and can therefore serve as a suitable surrogate in pre-clinical testing. Explant cultures of full-thickness retina preserved the complex intracellular processes and communications among the neural retinal cells thus demonstrating it as a useful model in target-tissue validation of AAV vector variants.

Briefly, eyes from healthy 6- to 7-month old American Yorkshire pigs were obtained from Surpass Inc. (Mountain View, Calif.) within 2 hours postmortem. After enucleation, the eyes were immediately placed in a sterile container and submerged in cold phosphate-buffered saline (PBS) solution. The eyes were kept on ice in a cooler and transported to Avalanche Biotechnologies (Menlo Park, Calif.).

Upon arrival, extraneous tissue surrounding the eye was removed using a scalpel and scissors. The eyeball was cut along the corneal limbus into anterior and posterior portions. The posterior segment (eyecup) was transferred to a petri dish containing CO₂-independent media (Gibco), and the vitreous humor was carefully removed leaving the neural retina attached to the eyecup. The optic disk was severed and the retina was subsequently peeled off and placed on to a 10 cm petri dish containing 5 mL of sterile, CO₂-independent media. 4-6 mm disposable circular biopsy punches were used to cut full thickness posterior segment retinal explants under a dissecting microscope. With the photoreceptor layer facing down, the retinal explant fragments were floated onto 150 μL CO₂-independent media on 12 mm Transwell® cell culture inserts with a 0.4 mm pore polycarbonate membrane (Corning). Once the retinal explants settled flat onto the Transwell® membrane, the CO₂-independent media was removed.

For culture of these explants, 0.5 mL of complete Neurobasal A (NBA) media supplemented with GlutaMAX, B-27 supplement, 100 units/mL of penicillin, 100 μg/mL streptomycin, and 0.25 μg/mL Amphotericin B (Gibco) was added into the well under the Transwell® insert to the level of the membrane such that the top surface of the tissue was exposed to the atmosphere. Explants were maintained at 37° C. in a 5% CO₂-humidified environment for up to 4 weeks with media changed every 48 hours.

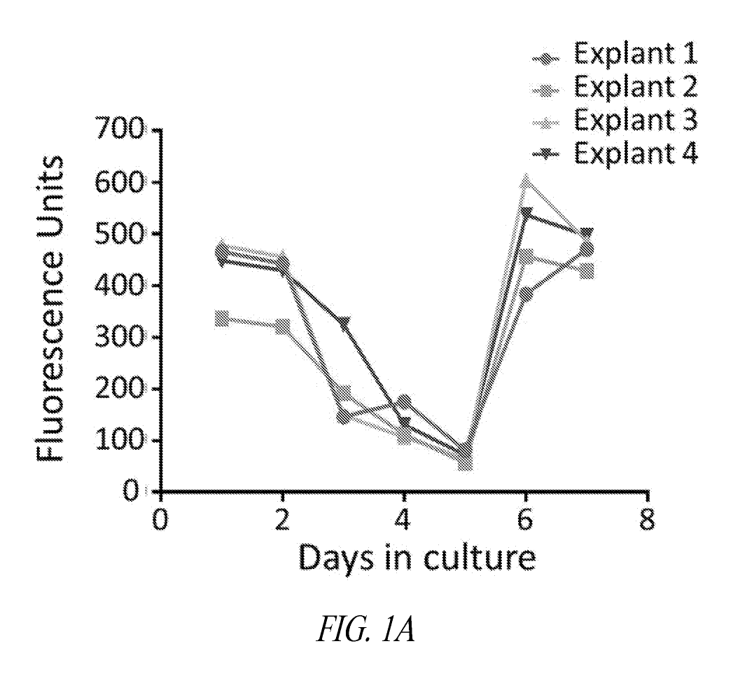

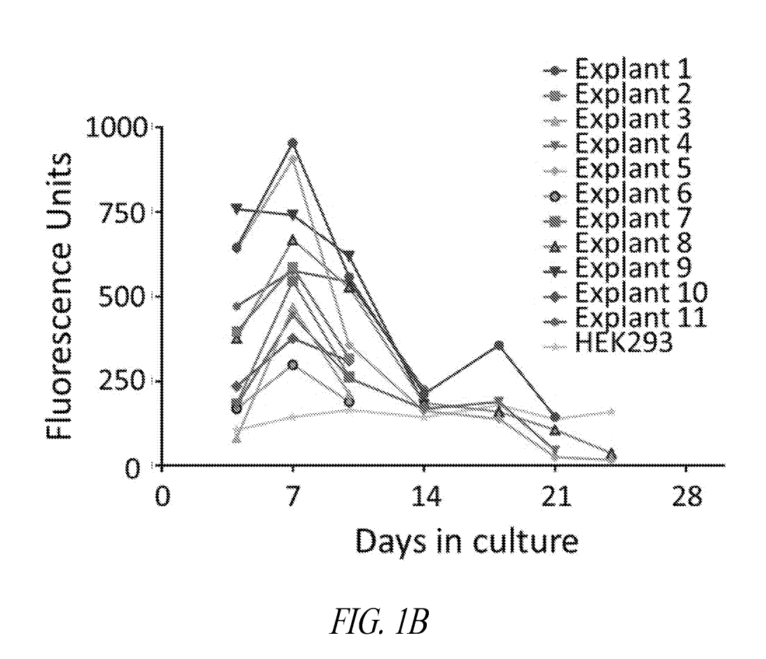

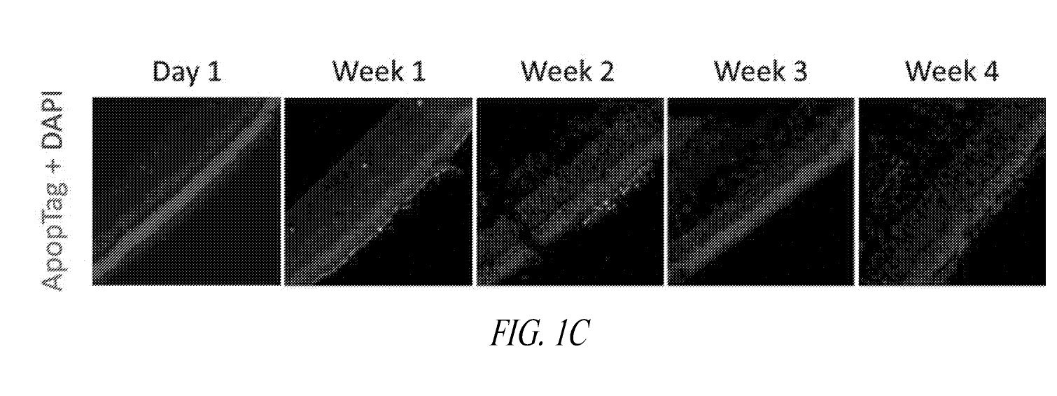

As shown in FIGS. 1A-1C, porcine retinal explants were viable in culture for up to 4 weeks. The release of glucose-6-phosphate dehydrogenase from dying cells was quantified daily during the first week of culture (FIG. 1A), and twice weekly for the remaining 2.5 weeks (FIG. 1B) as an indicator of cell viability. These data demonstrate a peak in cell death at week 1 followed by a steady decrease in death over time. TUNEL staining of transverse sections of retinal explants was also performed over a period of 4 weeks (FIG. 1C), and showed signs of apoptosis (indicated in green; ApopTag) only during the first 2 weeks of culture.

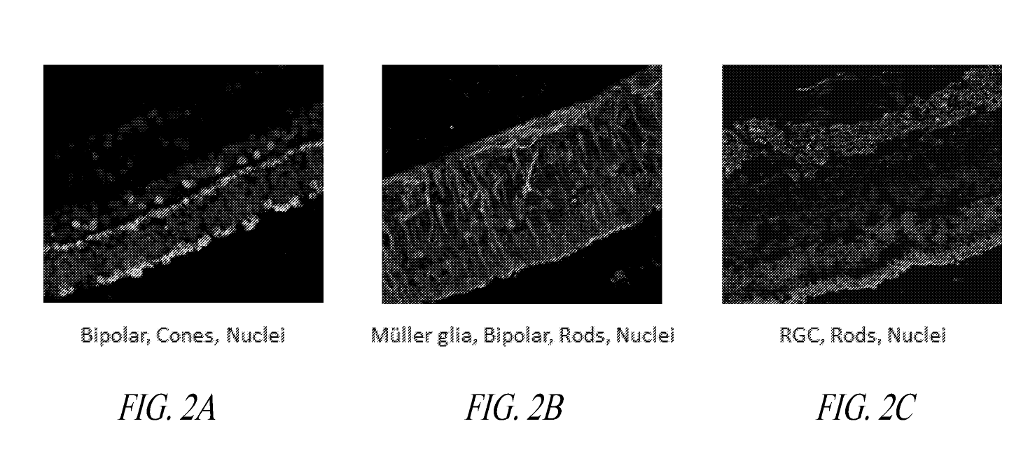

In addition to validating the cellular viability, cellular morphology and tissue architecture were examined. Immunofluorescence staining of retinal explants was performed to determine the preservation of neural retinal cell layers in culture. Transverse sections of explants were labeled with antibody markers for bipolar cells (CHX10), cone cells (L/M opsin), rod cells (rhodopsin), Muller glial cells (GFAP), and retinal ganglion cells (RGC, Tujl/β-tubulin 3) after 5 days in culture. FIG. 2A-FIG. 2C shows that the retinal explant culture system preserves most retinal cell types ex vivo, with tissue architecture and cell morphology closely resembling that of intact retinas in vivo.

### Example 2

### Protein Expression in Transduced Retinal Explant Cultures

Transductions with adeno-associated viral (AAV) vectors were performed on retinal explants the same day the cultures were started. AAV vectors were diluted in NBA media to obtain a 2E4 multiplicity of infection (MOI) per explant. For MOI calculations, the 4 mm retinal explant fragments were dissociated into single cell suspensions using the papain dissociated system (Worthington Biochemical Corporation) and all cells were counted. After the removal of the CO₂-independent media from the Transwell®, 2×10⁶ AAV-vector genomes were diluted in 100 μL NBA media and were added directly to each explant. Explants were then incubated in a 37° C., 5% CO₂ incubator for 2 hours. After 2 hours, 0.5 mL of complete NBA was added into the well under the Transwell® insert to the level of the membrane such that the top surface of the tissue was exposed to the atmosphere. Explants were maintained at 37° C. in a 5% CO₂-humidified environment for up to 4 weeks with media changed every 48 hours.

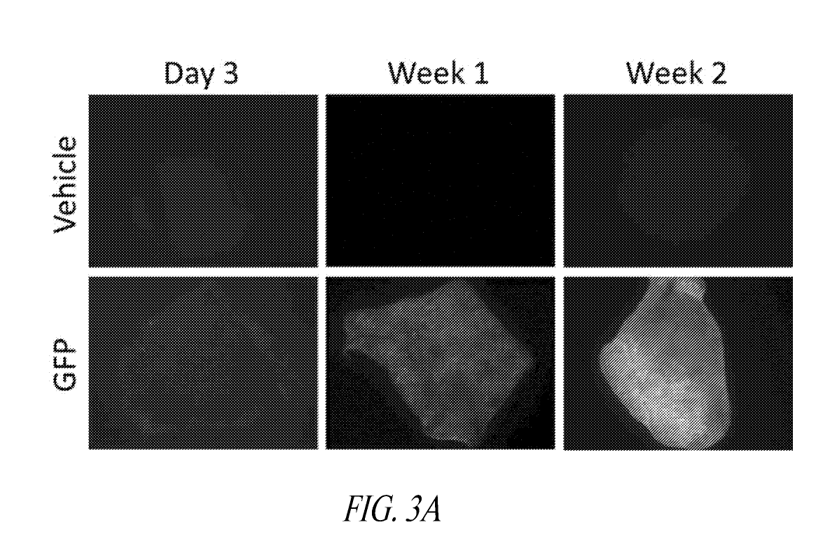

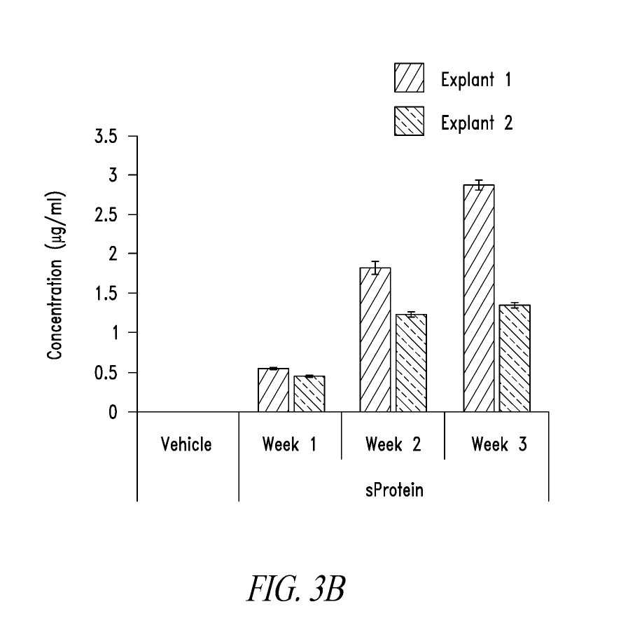

As shown in FIGS. 3A-3B, explants transduced with an AAV2.7m8 vector expressing GFP showed intracellular expression of the protein as early as day 3 post-transduction, with the GFP signal gradually increasing over time (FIG. 3A). Quantification of a secreted protein by ELISA showed a steady increase in expression over time (FIG. 3B). These data indicate that retinal explant cultures can be efficiently transduced, resulting in quick expression and secretion of functional proteins, with expression kinetics similar to those found in vivo.

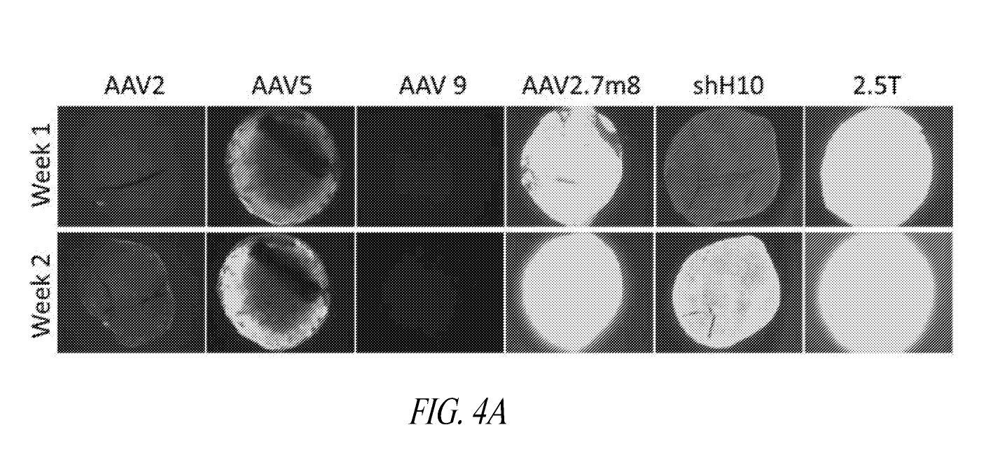

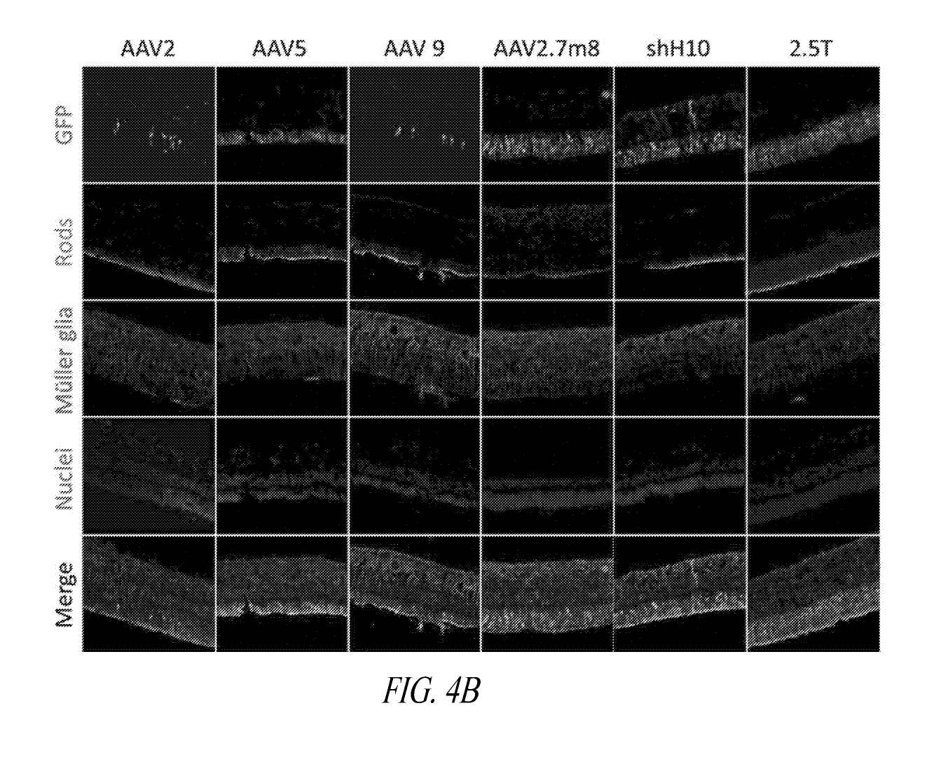

The protein levels induced by transduction with multiple AAV variants were also analyzed. Live cell imaging of GFP in retinal explants was performed at 1 and 2 weeks post-transduction with AAV2, AAV5, AAV9, AAV2.7m8, ShH10, and 2.5T variants (FIG. 4A). As shown, variants demonstrated variable expression levels of intracellular GFP. Further, immunofluorescence staining of transduced retinal explants 2 weeks post-transduction shows that different variants can also result in differential cellular expression of GFP in the retina (FIG. 4B).

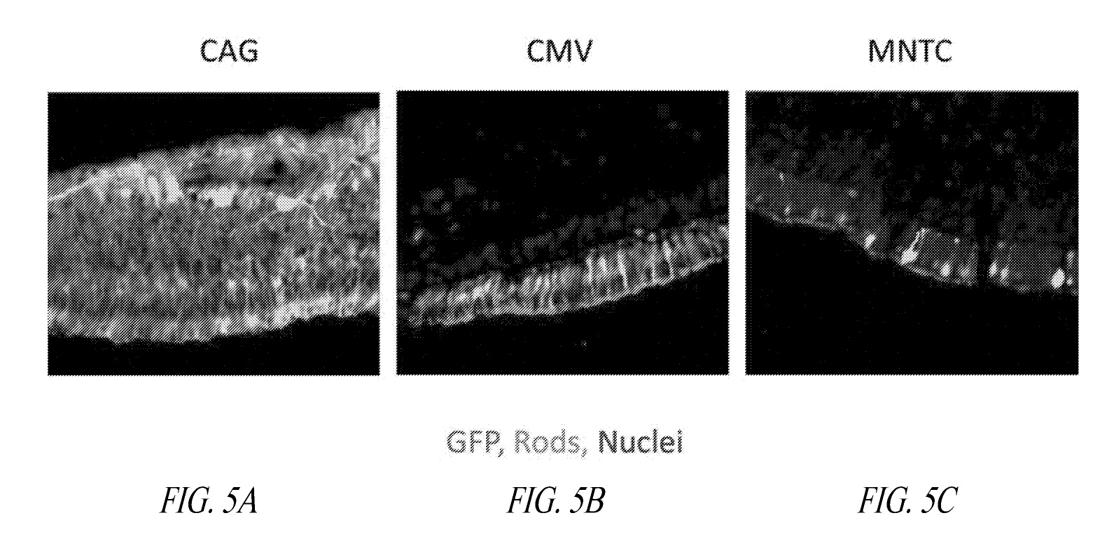

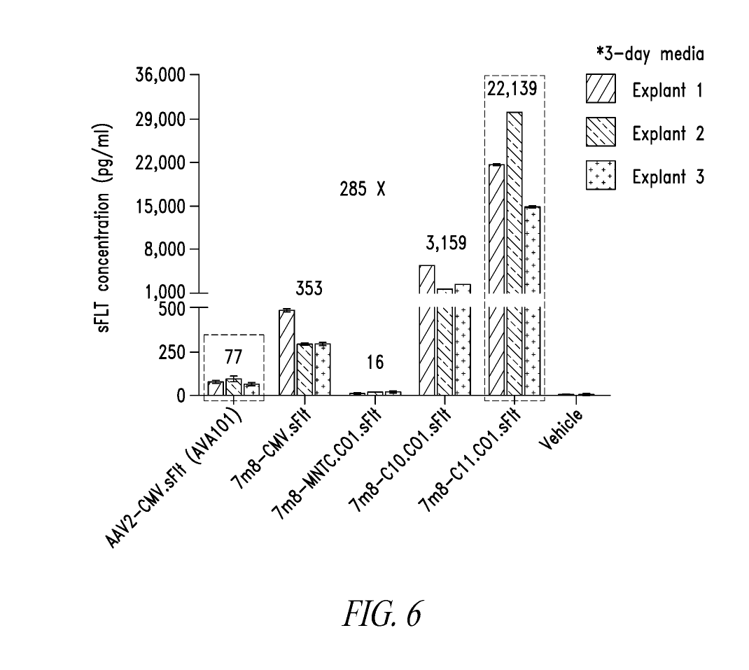

In addition to AAV-variants, various ubiquitous and cell-specific promoters were tested for their ability to induce GFP expression in retinal explant cultures (FIG. 5A-5C). The ubiquitous CAG promoter resulted in GFP expression throughout the retina (FIG. 5A), while the ubiquitous CMV promoter resulted in GFP expression that was more restricted (FIG. 5B). Further, the cone-specific promoter, MNTC, restricted expression to cone cells (FIG. 5C).

Secreted sVEGFR-1 protein levels were induced by transduction of with multiple AAV vectors (FIG. 6). Transduction with AAV2-CMV.sFlt (AVA101), 7m8-MNTC.CO1.sFlt, 7m8-C10.CO1.sFlt, and 7m8-C11.CO1. sFlt. Amounts of sVEGFR-1 present in supernatant were quantified by ELISA. As shown in FIG. 6, the 7m8-C11.CO1.sFlt construct dramatically increased the amount of sVEGFR1 proteins present in the supernatant. These results demonstrated that the organotypic retinal explant culture system can be used to evaluate effectiveness of different viral vectors for transfection into the retina.

These results demonstrate that multiple viral variants and multiple promoters can induce transgene expression in retinal explant cultures resulting in differential levels of overall protein expression and different cellular expression patterns. These results also highlight the importance of promoter and viral variant selection for modulating protein expression in transduced porcine retinal explants.

Overall, these data demonstrate that a porcine retinal explant model facilitate the evaluation of efficacy, cellular trophism, and promoter selectivity of rAAV vectors in the quick, reproducible, and economical manner and indicate that porcine retinal explants are a useful ex vivo screening system for AAV-mediated ocular gene therapy studies.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An organotypic culture system comprising a retinal explant comprising live cells and a cell culture medium; wherein the retinal explant is obtained from an eye from a pig or a non-human primate.

2. The organotypic culture system of claim 1, wherein the live cells are viable in the organotypic culture system for about two to about four weeks.

3. The organotypic culture system of claim 1 or 2, wherein tissue architecture of the retinal explant is preserved for about two to about four weeks.

4. The organotypic culture system of any of claims 1-3, wherein the live cells comprise a cell that expresses beta-III-tubulin, a cell that expresses Chx10, a cell that expresses rhodopsin, and/or a cell that expresses L/M opsin.

5. The organotypic culture system of any of claims 1-4, wherein the live cells comprise: ganglion cells, bipolar cells, cones, rods, retinal pigment epithelial cells, amacrine cells, horizontal cells, astrocytes, and/or Müller cells.

6. The organotypic culture system of any of claims 1-5, wherein the porcine retinal explant comprises an a nerve fiber layer, a ganglion cell layer, an inner plexiform layer, an inner nuclear layer, an outer plexiform layer, an outer nuclear layer, an external limiting membrane, and/or a layer of rods and cones.

7. The organotypic culture system of claim 6, wherein the retinal explant comprises all of: the nerve fiber layer, the ganglion cell layer, the inner plexiform layer, the inner nuclear layer, the outer plexiform layer, the outer nuclear layer, the external limiting membrane, and the layer of rods and cones.

8. The organotypic culture system of any of claims 1-5, wherein the non-human primate retinal explant comprises an inner a nerve fiber layer, a ganglion cell layer, an inner plexiform layer, an inner nuclear layer, an outer plexiform layer, an outer nuclear layer, an external limiting membrane, a layer of rods and cones and/or the retinal pigment epithelium.

9. The organotypic culture system of claim 6, wherein the retinal explant comprises all of: the nerve fiber layer, the ganglion cell layer, the inner plexiform layer, the inner nuclear layer, the outer plexiform layer, the outer nuclear layer, the external limiting membrane, the layer of rods and cones, and/or the retinal pigment epithelium.

10. The organotypic culture system of any of claims 1-9, wherein the retinal explant has a diameter of between about 4 mm to about 16 mm.

11. The organotypic culture system of any of claims 1-10, wherein the retinal explant is obtained from an adult pig or an adult non-human primate.

12. The organotypic culture system of any of claims 1-11, wherein the porcine retinal explant is obtained from a pig that is about six to about seven months old.

13. The organotypic culture system of any of claims 1-12, wherein the porcine retinal explant is obtained from an American Yorkshire pig.

14. The organotypic culture system of any of claims 1-13, wherein the organotypic culture system further comprises a transwell containing the retinal explant, and wherein the transwell is inserted into a well containing the cell culture medium.

15. The organotypic culture system of claim 14, wherein the transwell comprises a top compartment, wherein the top compartment is composed of a circular wall and a porous membrane that forms the base of the top compartment, wherein the porous membrane comprises a top surface and a bottom surface, wherein the retinal explant is contained in the top compartment and contacts the top surface of the membrane; and wherein the bottom surface of the membrane contacts the cell culture medium.

16. The organotypic culture system of claim 15, wherein the retinal explant comprises a surface comprising an exposed photoreceptor layer or an exposed retinal pigment epithelium and a surface comprising an exposed nerve fiber layer, and wherein the face comprising the exposed photoreceptor cell layer or exposed retinal pigment epithelium contacts the top surface of the membrane.

17. The organotypic culture system of claim 16, wherein the surface comprising the exposed photoreceptor layer or the exposed retinal pigment epithelium is exposed to the cell culture medium, and the surface comprising the exposed nerve fiber layer is exposed to atmosphere.

18. The organotypic culture system of any of claims 1-17, wherein the cell culture medium comprises Neurobasal A.

19. The organotypic culture system of any of claims 1-18, wherein the cell culture medium comprises an L-glutamine supplement, a neuron supplement, and at least one antibiotic.

20. The organotypic culture system of claim 19, wherein the L-glutamine supplement is GlutaMAX, the neuron supplement is B-27, and the at least one antibiotic is a combination of penicillin, streptomycin, amphotericin B, and a growth factor.

21. The organotypic culture system of any of claims 1-20, wherein a plurality of the live cells comprise an exogenous polynucleotide.

22. The organotypic culture system of claim 21, wherein the exogenous polynucleotide is an expression vector, an mRNA, an expression cassette, or an oligonucleotide.

23. The organotypic culture system of claim 22, wherein the expression vector is a virus, optionally an adeno-associated virus (AAV), an adenovirus, or a lentivirus.

24. A method of determining expression of an exogenous polynucleotide in a retinal explant, comprising:
(A) isolating the retinal explant from an eye of a pig or a non-human primate, wherein the portion of the retinal explant comprises live cells;
(B) contacting the retinal explant with the exogenous polynucleotide;
(C) incubating the retinal explant in an organotypic culture system for a period of time; and
(D) determining the expression of the exogenous polynucleotide in the retinal explant;
thereby determining the expression of the exogenous polynucleotide;
wherein the retinal explant is a porcine retinal explant or a non-human primate retinal explant.

25. The method of claim 24, wherein the exogenous polynucleotide is an expression vector, an mRNA, an expression cassette, or an oligonucleotide.

26. The method of claim 25, wherein the expression vector is a virus, optionally an adeno-associated virus (AAV).

27. The method of any of claims 24-26, wherein step (A) comprises the steps of
(i) obtaining the eye from the pig or non-human primate;
(ii) isolating a retina from the eye; and
(iii) removing a portion from the retina;
thereby isolating the retinal explant from the eye.

28. The method of claim 27, wherein step (A)(i) comprises enucleating the eye from the pig or non-human primate within two hours postmortem and placing the eye in cold phosphate buffered saline (PBS).

29. The method of any of claims 24-28, wherein the pig or non-human primate is an adult animal.

30. The method of claim 29, wherein the adult pig is about six to about seven months old.

31. The method of any of claims 24-30, wherein the pig is an American Yorkshire pig.

32. The method of any of claims 27-31, wherein the step of (A)(ii) comprises the steps of:
(a) cutting the eye along the corneal limbus of the eye into an anterior portion and a posterior portion, wherein the posterior portion comprises an optic disc and the retina;
(b) placing the posterior portion into a dissection medium;
(c) removing the retina from the posterior portion;
thereby isolating the retina from the eye.

33. The method of claim 32, wherein extraneous tissue is removed from the eye prior to performing the step of (A)(ii)(a).

34. The method of claim 32 or 33, wherein the dissection medium is a CO₂-independent medium.

35. The method of any of claims 32-34, wherein the optic disc of the posterior portion is severed prior to performing the step of (A)(ii)(c).

36. The method of any of claims 27-35 wherein step (A)(iii) comprises cutting the retina with a circular biopsy punch, thereby obtaining the retinal explant.

37. The method of any of claims 24-36, wherein the retinal explant has a diameter of about 4 mm to about 6 mm.

38. The method of any of claims 24-37, wherein the porcine retinal explant comprises a nerve fiber layer, a ganglion cell layer, an inner plexiform layer, an inner nuclear layer, an outer plexiform layer, an outer nuclear layer, an external limiting membrane, and/or a layer of rods and cones.

39. The method of any of claims 24-37, wherein the porcine retinal explant comprises all of: the nerve fiber layer, the ganglion cell layer, the inner plexiform layer, the inner nuclear layer, the outer plexiform layer, the outer nuclear layer, the external limiting membrane, and the layer of rods and cones.

40. The method of any of claims 24-37, wherein the non-human primate retinal explant comprises wherein the non-human primate retinal explant comprises an inner a nerve fiber layer, a ganglion cell layer, an inner plexiform layer, an inner nuclear layer, an outer plexiform layer, an outer nuclear layer, an external limiting membrane, a layer of rods and cones and/or the retinal pigment epithelium.

41. The method of any of claims 24-37, wherein the non-human primate retinal explant wherein the retinal explant comprises all of: the nerve fiber layer, the ganglion cell layer, the inner plexiform layer, the inner nuclear layer, the outer plexiform layer, the outer nuclear layer, the external limiting membrane, the layer of rods and cones, and the retinal pigment epithelium.

42. The method of any of claims 24-41, wherein step (B) comprises the steps of;
(i) placing the retinal explant in a cell culture dish, in a well of a cell culture plate, or in a transwell that is contained in a well of a cell culture plate; and
(ii) contacting the porcine retinal explant with a solution comprising the exogenous polynucleotide; and
thereby contacting the portion of the retina to a exogenous polynucleotide.

43. The method of claim 42, wherein the transwell comprises a top compartment, wherein the top compartment is composed of a circular wall and a porous membrane that forms the base of the top compartment, wherein the porous membrane comprises a top surface and a bottom surface, and wherein step (B)(i) comprises placing the porcine retinal explant in the top compartment of the transwell.

44. The method of claim 43, wherein the retinal explant comprises a surface comprising an exposed photoreceptor layer or an exposed retinal pigment epithelium and a surface comprising an exposed nerve fiber layer, and wherein the face comprising the exposed photoreceptor cell layer or exposed retinal pigment epithelium is placed in the top compartment of the transwell and contacts the top surface of the membrane.

45. The method of claim 44, wherein the face comprising the exposed photoreceptor layer or the exposed retinal pigment epithelium is exposed to the cell culture medium, and the surface comprising the exposed nerve fiber layer is exposed to atmosphere.

46. The method of any of claims 43-45, wherein step (B)(ii) is performed by placing the solution into the top compartment of the transwell, thereby contacting the exogenous polynucleotide to the porcine retinal explant.

47. The method of any of claims 24-46, wherein the exogenous polynucleotide is a virus and the porcine retinal explant is contacted with virus in an amount of about 1×10³ to about 1×10⁵ multiplicity of infection (MOI).

48. The method of claim 47, wherein the amount is about 2×10⁴ MOT.

49. The method of any of claims 24-46, wherein the exogenous polynucleotide is a virus, and wherein the porcine retinal explant is contacted with the virus at a concentration of about 1×10⁴ to about 1×10¹² polynucleotide genomes per microliter.

50. The method of claim 49, wherein the concentration is about 2×10⁸ vector genomes per microliter.

51. The method of any of claims 24-50, wherein the exogenous polynucleotide contacts the porcine retinal explant for about 15 minutes to about 12 hours.

52. The method of claim 51, wherein the exogenous polynucleotide contacts the porcine retinal explant for about 2 hours.

53. The method of any of claim 24-39 or 47-52, wherein step (C) comprises the steps of:
(i) placing the retinal explant in a cell culture dish, a well of a cell culture plate, or a transwell that is contained in a well of a cell culture plate; and
(ii) adding an organotypic culture medium to the cell culture dish, the well of the cell culture plate, or the transwell.

54. The method of claim 53, wherein the transwell comprises a top compartment, wherein the top compartment is composed of a circular wall and a porous membrane that forms the base of the top compartment, wherein the porous membrane comprises a top surface and a bottom surface, and wherein step (C)(i) comprises placing the retinal explant in the top compartment of the transwell,
and wherein step (C)(ii) comprises adding an organotypic culture medium to the well containing the transwell, wherein the organotypic culture medium is added to the height of the membrane of the transwell so that the organotypic culture medium contacts the bottom surface of the porous membrane.

55. The method of claim 54, wherein the retinal explant comprises a surface comprising an exposed photoreceptor layer or an exposed retinal pigment epithelium and a surface comprising an exposed nerve fiber layer, and wherein the surface comprising the exposed photoreceptor cell layer or exposed retinal pigment epithelium is placed in the top compartment of the transwell and contacts the top surface of the membrane, and wherein the face comprising the exposed photoreceptor layer or the exposed retinal pigment epithelium is exposed to the cell culture medium, and the surface comprising the exposed nerve fiber layer is exposed to atmosphere.

56. The method of any of claims 42-52, wherein step (C) comprises adding an organotypic culture medium into a well containing the transwell, wherein the organotypic culture medium is added to the height of the membrane of the transwell so that the organotypic culture medium contacts the bottom surface of the porous membrane, and so that the organotypic culture medium contacts the porcine retinal explant at the surface exposing the retinal pigment epithelium and does not contact the porcine retinal explant at a surface exposing the inner limiting membrane.

57. The method of any of claims 53-56, wherein the organotypic culture medium comprises Neurobasal A media.

58. The method of any of claims 53-57, wherein the organotypic retina cell culture medium comprises an L-glutamine supplement, a neuron supplement, at least one antibiotic, and/or at least one growth factor.

59. The method of claim 58, wherein the L-glutamine supplement is GlutaMAX, the neuron supplement is B-27, and the at least one antibiotic is a combination of penicillin, streptomycin, and amphotericin B.

60. The method of any of claims 24-59, wherein the period of time is between about 3 days and about 42 days.

61. The method of claim 60, wherein the period of time is between about 14 days and 28 days.

62. The method of any of claims 24-61, wherein at least a portion of cells of the porcine retinal explant are viable during the period of time.

63. The method of any of claims 24-62, wherein the exogenous polynucleotide encodes a polypeptide.

64. The method of claim 63, wherein step (D) comprises detecting the polypeptide.

65. The method of claim 64, wherein the polypeptide is a therapeutic polypeptide.

66. The method of claim 65, wherein the polypeptide is a fluorescent polypeptide.

67. The method of claim 66, wherein the fluorescent polypeptide comprises green fluorescent protein (GFP).

68. The method of claim 64, wherein the polypeptide is a secreted polypeptide.

69. The method of claim 68, wherein the secreted polypeptide is VEGFR-1 or another protein that binds to VEGF.

70. The method of any of claims 64-69, wherein the polypeptide is detected by live cell imaging, immunofluorescence, western blotting, and/or ELISA.

71. The method of any of claims 64-70, wherein detecting the polypeptide further comprises labeling a population of cells of the porcine retinal explant.

72. The method of claim 71, wherein labeling the population of cells of the porcine retinal explant comprises contacting the population with an antibody that recognizes a marker of the population of cells, and detecting the antibody with immunofluorescence.

73. The method of claim 71 or 72, wherein the population of cells is selected from the group consisting of: ganglion cells, bipolar cells, cones, rods, retinal pigment epithelial cells, amacrine cells, horizontal cells, astrocytes, and Müller cells.
